(19) United States
(12) Patent Application Publication
(10) Pub. No.: US 2025/0295886 A1
(43) Pub. Date: Sep. 25, 2025

(54) GUIDE EXTENSION CATHETER

(71) Applicant: Teleflex Life Sciences LLC, Wilmington, DE (US)

(72) Inventors: Joshua Brenizer, Oak Grove, MN (US); Christopher E. Buller, Toronto (CA); Dean Peterson, Minneapolis, MN (US); Allison Margulies, Minneapolis, MN (US)

(73) Assignee: Teleflex Life Sciences LLC, Wilmington, DE (US)

(21) Appl. No.: 19/083,180

(22) Filed: Mar. 18, 2025

Related U.S. Application Data
(60) Provisional application No. 63/567,373, filed on Mar. 19, 2024.

(51) Int. Cl. A61M 25/00; A61M 25/06

(52) U.S. Cl. CPC A61M 25/0023; A61M 25/0668; A61M 2025/0024; A61M 2025/0681

(57) ABSTRACT

A guide extension catheter positionable within a guide catheter and configured to receive an interventional device for insertion into a vasculature is disclosed. The guide extension catheter can include a push member and a radially collapsible, tubular membrane in contact with the push member. The guide extension catheter may include a reinforcement member or proximal end cap positioned at the proximal end of the tube member and formed as a rigid, non-collapsible structure. The guide extension catheter may be delivered through another catheter, such as a guide catheter, in a compressed configuration, and then released to an expanded, non-compressed configuration when the guide extension catheter has been partially extended beyond a distal end of the guide catheter.

### CROSS-REFERENCE TO RELATED APPLICATIONS

This application claims the benefit of priority under 35 U.S.C. § 119 (c) to U.S. Provisional Appln. Ser. No. 63/567,373, filed Mar. 19, 2024, which is incorporated herein by reference in its entirety.

### TECHNICAL FIELD

The subject matter of this disclosure relates to the field of medical devices. Implementations relate to guide extension catheters and components thereof.

### BACKGROUND

The present disclosure relates generally to devices, systems, and methods for interventional procedures, and more particularly to a guide extension catheter for aiding in the delivery of interventional devices to a treatment site within a patient.

In general, interventional procedures require delivering interventional devices though guide catheters. It is often necessary to deliver the interventional device to a desired location beyond a distal end of the guide catheter, i.e., a target tissue area, for the device to administer an effective treatment. However, delivery of the interventional device beyond the guide catheter may require high delivery force and can cause micro and/or macro injuries to vasculature en route to the target tissue area.

### SUMMARY

The present inventors recognize that there exists a need for catheter delivery devices, systems, and methods which can be used to deliver an interventional device to a desired location and shield the vasculature from abrasion or injury.

Embodiments as described in this disclosure include a guide extension catheter. The guide extension catheter may include a proximal elongate shaft; and a distal, self-expanding elongate sheath member coupled with the elongate shaft, the elongate sheath member expandable between a radially contracted configuration and a radially expanded configuration and defining a passageway. The elongate sheath member in the radially contracted configuration may have an outer profile no more than three times greater than an outer profile of the elongate shaft, and the elongate sheath member in the radially expanded configuration may have an outer profile at least four times greater than the outer profile of the elongate shaft.

Embodiments as described in this disclosure include a method. The method may involve advancing a distal end of a predefined length guide catheter having a continuous lumen through a blood vessel to an ostium of a coronary artery; advancing a distal end portion of a guide extension catheter through and beyond the distal end of the guide catheter, including advancing an elongate shaft eccentrically coupled with a self-expanding elongate sheath member in a radially-contracted configuration through and beyond the distal end of the guide catheter; displacing an outer delivery cover surrounding the elongate sheath member, thereby allowing the elongate sheath member to expand from the radially-contracted configuration to a radially-expanded configuration defining a passageway having a diameter greater than or equal to 0.056 inches; and while maintaining the distal end portion of the guide extension catheter beyond the distal end of the guide catheter, advancing an interventional device at least partially through the continuous lumen of the guide catheter, into and through the passageway of the elongate sheath member, and into the coronary artery.

The guide extension catheters as described herein can provide a low friction, large diameter (for a given compatible guide catheter size) pathway proximal to, and optionally through, a target lesion in the vasculature, thereby reducing micro and macro vasculature injury attributable to delivery of interventional devices. Related systems and methods are also disclosed. These and other examples and features of the present devices, systems, and methods will be set forth, at least in part, in the following Detailed Description. This Summary is intended to provide non-limiting examples of the present subject matter-it is not intended to provide an exclusive or exhaustive explanation. The Detailed Description below is included to provide further information about the present devices, systems, and methods.

### BRIEF DESCRIPTION OF DRAWINGS

This written disclosure describes illustrative embodiments that are non-limiting and non- exhaustive. Reference is made to illustrative embodiments that are depicted in the figures, in which:

FIG. 1 illustrates a plan view of a guide catheter advanced through an aorta to an ostium of a coronary vessel, according to some embodiments.

FIG. 2 illustrates a plan view of a guide extension catheter, as constructed in accordance with at least one embodiment, used in conjunction with a guide catheter for the delivery of an interventional device into an occluded vessel for treatment.

FIG. 3 illustrates a partial cutaway view of a guide extension catheter, according to some embodiments.

FIGS. 3A-3C illustrate views of configurations of the coil loops included in a guide extension catheter of FIG. 3, according to some embodiments.

FIG. 3D illustrates a cross-sectional view of a portion of the guide extension catheter of FIG. 3, according to some embodiments.

FIG. 3E illustrates an end portion of the guide extension catheter of FIG. 3, according to some embodiments.

FIGS. 4A-4D illustrate partial cutaway views of various stages of deployment of the guide extension catheter of FIG. 3.

FIG. 4E illustrates a partial cutaway view of the guide extension catheter of FIG. 3 deployed in a guide catheter and receiving a treatment device, according to various embodiments.

FIG. 5 illustrates a partial cutaway view of a guide extension catheter, according to some embodiments.

FIGS. 6A-6D illustrate partial cutaway views of various stages of deployment of the guide extension catheter of FIG. 5.

FIG. 6E illustrates a partial cutaway view of the guide extension catheter of FIG. 5 deployed in a guide catheter and receiving a treatment device, according to various embodiments.

FIG. 7 illustrates a flowchart of a method for accessing a coronary artery and providing treatment to the artery, according to some embodiments.

FIG. 8A illustrates an isometric view of a portion of a guide catheter and a guide extension catheter having a distal reinforcement member, according to some embodiments.

FIG. 8B illustrates a magnified isometric view of a distal portion of the guide extension catheter shown in FIG. 8A, according to some embodiments.

FIG. 8C illustrates a side view of an interventional device being retracted proximally toward a distal end of a configuration of the guide extension catheter shown in FIG. 8A, according to some embodiments.

FIG. 8D illustrates a side view of an interventional device being retracted proximally toward a distal end of another configuration of the guide extension catheter shown in FIG. 8A, according to some embodiments.

### DETAILED DESCRIPTION

According to some embodiments, this disclosure relates to a guide extension catheter having a push member and a radially collapsible tubular membrane (tubular member) having a lumen coupled to the push member. In various embodiments, the tubular member includes a reinforcement portion that extends along a portion of the tubular member, the reinforcement portion formed from a rigid, non-collapsible material, and forms a portion of the lumen extending through the tubular member. The tubular membrane may have minimal effective radial strength in compression, minimal effective column strength, and minimal effective bend stiffness. The tubular membrane may have sufficient tensile strength to avoid tearing during insertion of an interventional device and during its removal from a patient. The tubular membrane may be durably lubricious on an inner surface to facilitate advancement and withdrawal of interventional devices through its lumen and may be durably lubricious on an outer surface to enhance delivery of the guide extension catheter into a blood vessel. In various embodiments, the tubular member includes a plurality of coil loops attached to the push member and spaced along the push member, which are enclosed by an elongate sheath formed from a flexible and radially collapsible material. The coil loops are configured to extend and expand open the lumen formed by the elongate sheath when an outer delivery cover that encircles the elongate sheath and the coil loops, and which holds the coil loops and the elongate sheath in a radially compressed configuration, is removed. In various embodiments, the outer delivery cover is removed either just before or just after the tubular member of the guide extension catheter is partially extended beyond the distal end of an outer or guide catheter where the tubular member is positioned. Once the outer delivery layer has been removed, the coil loops act as spring elements to extend and open the portions of elongate sheath extending along the tubular member, and to form a cavity extending through the tubular member that can accommodate the delivery of the treatment device through the lumen.

According to some embodiments, the push member may serve as a backbone to the tubular membrane. The push member may optionally be in the form of a guidewire or a push rod, which may be a gradually tapering push rod, to help steer and support delivery of the guide extension catheter to a target tissue area. The reinforcement portion of the guide extension catheter may be disposed at a proximal end of the tubular membrane and may provide structural support to keep the proximal end of the tubular membrane's lumen open and accessible. The guide extension catheter may further include a second reinforcement portion disposed at a distal end of the tubular membrane, which is configured to keep the distal end of the tubular membrane's lumen open. In some examples, the second reinforcement portion disposed at a distal end of the tubular membrane is configured to pivot, lean, tilt, or otherwise undergo a change in angular position upon interacting with another device (e.g., a treating catheter) advanced or retracted coaxially. For example, the second, distal reinforcement portion may undergo a change in angular orientation upon contacting a proximally retracting interventional device in a manner that opens the passageway for the device defined by the lumen of the tubular membrane. A change in angular orientation may also occur upon contacting a distally advancing interventional device. According to such embodiments, the second, distal reinforcement member may comprise a resilient support ring or coil member having a flexible, deformable, shape- memory material and/or resilient configuration, which may be biased toward a resting-state configuration.

Devices, systems, and methods herein relate generally to delivery of medical treatment devices through a guide extension catheter, and more specifically to devices, systems, and methods for enhanced and atraumatic delivery of interventional devices in patients undergoing percutaneous interventions in order to (i) deliver interventional devices that may not be easily delivered with a chosen in-situ guide catheter alone, and/or (ii) reduce micro and macro arterial injury attributable to delivery of inflexible or non-lubricious interventional devices and existing guide extension catheters. It should be noted that although the description below is primarily directed toward cardiovascular percutaneous interventions, the devices, systems, and methods described herein may be used in other medical specialties, e.g., peripheral vasculature treatments, urinary treatments, respiratory treatments, digestive treatments, diagnostic endoscope treatments, and/or any other medical treatments that can benefit from the use of guide extension catheters.

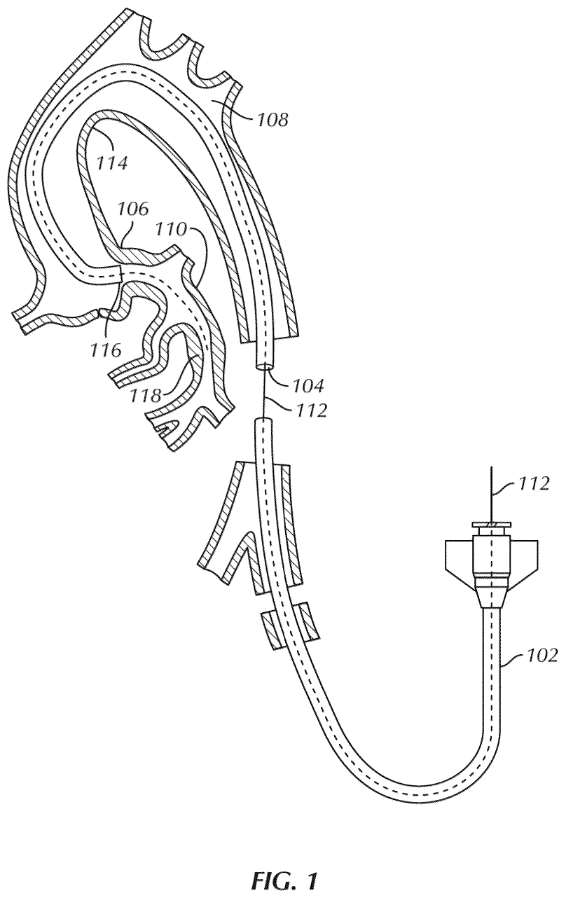

FIG. 1 illustrates an exemplary minimally invasive cardiac intervention, including a guidewire 112 and a guide catheter 102. The guidewire 112 can comprise an elongate, small-diameter member designed to navigate vessels to reach a diseased site or vessel segment of interest. Guidewires can come in various configurations, including stainless steel or nitinol core wires and/or solid core wires wrapped in a smaller wire coil, for example. The guide catheter 102 can comprise an elongate tube member defining a main lumen 104 along its length. The guide catheter 102 can be formed of polyurethane, for example, and can be shaped along its distal portion to facilitate advancement to and alignment with a coronary ostium 106 (or other region of interest within a patient's body). Variously sized guide catheters 102, e.g., 6 F, 7 F, or 8 F guide catheter, where F is an abbreviation for the French catheter scale (a unit to measure catheter diameter (1 F=⅓ mm)), can be inserted at a femoral or radial artery and advanced through an aorta 108 to a position adjacent the ostium 106 of a coronary artery 110.

The guidewire 112 (or a shorter, thicker introducer guidewire) and guide catheter 102 can be advanced through the arch 114 of the aorta 108 to the ostium 106. The guidewire 112 may then be advanced beyond the ostium 106 and into the coronary artery 110. The diameter and rigidity of the guide catheter's distal end 116, however, may not permit the device to be safely advanced beyond the ostium 106 into the coronary artery 110.

Maintaining the position of the guide catheter's distal end 116 at the ostium 106 can facilitate the guidewire 112, or another interventional device, successfully reaching the diseased site (e.g., a stenotic lesion 118). With the guide catheter 102 in position, force can be applied to the guidewire's proximal end to push the guidewire 112 to and beyond the lesion 118, and a treating catheter (optionally including a balloon or stent) can be passed over the guidewire 112 to treat the site. However, the application of force to the guidewire 112 or the treating catheter can sometimes cause the guide catheter 102 to dislodge from the ostium 106 of the coronary artery 110, and, in such instances, the guidewire or treating catheter must be distally advanced independently of the guide catheter's ostial alignment and support to reach the lesion 118. This can occur in the case of a tough stenotic lesion 118 or tortuous anatomy, for example, where it is often difficult to pass the guidewire 112 or the treating catheter to and beyond the lesion. A heart's intrinsic beat can also cause the guide catheter's distal end 116 to lose its ostial positioning or otherwise be shifted so that it no longer is positioned to align and support the guidewire 112 or the treating catheter into the portion of the coronary artery 110 including the lesion 118.

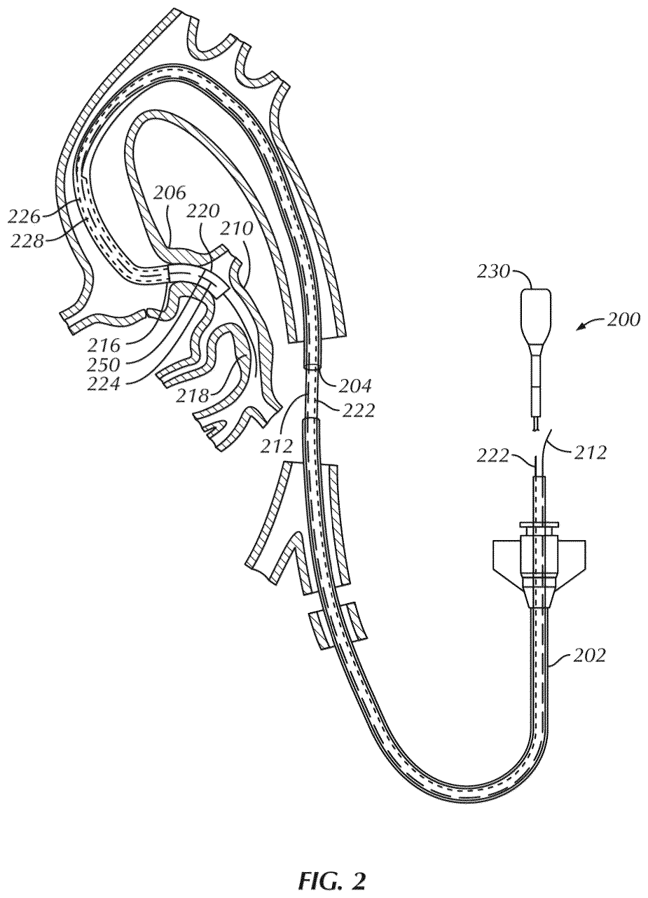

As first illustrated in FIG. 2, the present guide extension catheter 200 can improve access and provide protection to a coronary artery 210 leading up to, and optionally beyond, a stenotic lesion 218. The guide extension catheter 200 may include an elongate tube member 220 and a push member 222 having a collective length that is greater than a length of a guide catheter 202 (e.g., 130 cm-175 cm, or greater). An outer diameter of the tube member 220 can be sized to permit insertion of its distal end 224 through a guide catheter 202 and into the coronary artery 210 or its branches containing the lesion 218, thereby providing alignment, support, and a low-friction pathway for an interventional device (e.g., a treating catheter) beyond the distal end 216 of the guide catheter 202 to, and optionally through, the lesion 218. The extension of the tube member 220 into a smaller-sized artery or branch can also serve to maintain the position of the guide catheter 202 at the artery's ostium 206 during an operation.

The push member 222 may be in the form of a guidewire or a push rod, e.g., a gradually tapering push rod, for example, to help steer and support delivery of the guide extension catheter 200 to the lesion 218. The push member 222 may comprise a stainless steel, nitinol, and/or another rigid or substantially rigid material and can be configured to be sufficiently rigid in torque to avoid helical twisting of the guide extension catheter 200 during use. Generally, the push member 222 may comprise an elongate portion of the guide extension catheter that is rigid enough to push the device through a guide catheter, for example upon a user manually urging the push member in a distal direction. In various embodiments, the push member 222 may be flattened in cross section along one or more portions of its length to contribute to resistance to twisting and reduce a crossing profile of the guide extension catheter 200. Examples of the push member 222 may feature a variety of cross-sectional shapes and sizes, which may remain constant or may change along the length of the push member. Certain embodiments of the push member 222 may be similar or the same as the examples described in commonly owned U.S. Pat. Pub. No. 2019/0247619, which is hereby incorporated by reference in its entirety, including push members 422 and 522 disclosed therein. Embodiments may feature a push member having an arcuate surface configured to match or substantially match the curvature of the guide catheter, as described in commonly owned U.S. Pat. No. 10,751,514, which is hereby incorporated by reference in its entirety.

The tube member 220 may include a first reinforced portion (not shown) disposed at its proximal end 226 and a second reinforced portion disposed at its distal end 224. The tube member 220 may further include a soft, flexible, radially collapsible tubular membrane 250 disposed distally to the first reinforced portion and proximally to the second reinforced portion. In embodiments, the tubular membrane 250 may be connected to, and may overlap with, the first reinforced portion and/or the second reinforced portion.

The delivery of inflexible or non-lubricious interventional devices through a segment of the coronary artery 210 distal to the guide catheter 202 in the absence of the guide extension catheter 200 can produce (i) endothelial injury (micro injury) and may contribute to atheroembolism and type-4 periprocedural myocardial infarction, and/or (ii) more serious macro injuries including plaque disruption and coronary dissection leading to acute/threatened ischemic complication-any of which can contribute to atherosclerosis progression and eventual target-vessel failure. The soft, flexible, radially collapsible tubular membrane 250 can reduce device-artery interactions by providing a thin-walled structure that, once partially extended from the distal end of the guide catheter and allowed to expand to the uncompressed configuration, form a lumen that lines a portion the coronary artery 210 and provides a lubricious intra-coronary delivery pathway.

In some embodiments, the operating physician can advance the distal end portion 224 of the tube member 220 over a guidewire 212 and through and beyond the guide catheter's distal end 216 into the coronary artery 210 by applying a longitudinal force to the push member 222 directly or via a handle member 230, such as the handle member 230 described in commonly owned U.S. Pat. Pub. No. 2019/0247619, which is hereby incorporated by reference in its entirety. The handle member 230 may include a flexible clip or clamp configured to attach to an external object when not being moved, as described in commonly owned U.S. Pat. Pub. No. 2021/0008342, which is hereby incorporated by reference in its entirety. The proximal end portion 226 of the tube member 220 may remain within the guide catheter 202 during a procedure. The physician can subsequently deliver a treating catheter over the guidewire 212, through a main lumen 204 of the guide catheter 202, and through a lumen 228 of the tube member 220 until the working portion of the treating catheter is located beyond the distal end 224 of the tube member. Through use of the tube member 220, the operating physician can shield the vasculature from abrasion or injury caused by advancement of the treating catheter toward the lesion 218. Additionally, the tube member 220 can provide added alignment support to the guide catheter 202 relative to the coronary ostium as the treating catheter is advanced.

In general, the lumen 228, and hence the tube member 220 when expanded, can be sized and shaped to pass one or more interventional devices such as the guidewire and the treating catheter therethrough. The cross-sectional shape of the expanded lumen 228 can be similar to the cross-sectional shape of the guide catheter's main lumen 204. For instance, in some examples, the cross-sectional shape of the expanded lumen 228 can be generally uniform along its length. In other examples, the cross-sectional diameter may vary along the length of the tube member 220. According to embodiments of such examples, the distal end 224 of the tube member 220 may be narrower, e.g., tapered, relative to the proximal end 226, for instance. The length of each differently sized portion of the tube member 220 in such embodiments can also vary, and in some examples, the distal end 224 of the tube member can be the longest. In examples that include differently sized proximal and distal ends, the difference in diameter between the proximal end 226 and the distal end 224 of the tube member may be from about 1 F to about 4 F, or anywhere in between.

The outer diameter of the tube member 220 when expanded can assume maximum cross-sectional dimensions that allow the tube member 220 to coaxially slide relative to the guide catheter 202. In other embodiments, the outer cross-sectional dimensions of the tube member 220 when expanded can be less than the allowable maximum. In varying embodiments, a diameter of the lumen 228 of the tube member 220 when expanded is not more than about one French size smaller than a diameter of the lumen 204 of the guide catheter 202. In one embodiment, the guide extension catheter 200 can be made in at least three sizes corresponding to the internal capacity of 8 F, 7 F, and 6 F guide catheters that are commonly used in interventional cardiology procedures. The difference in size between the outer diameter of the tube member 220 when expanded and the inner diameter of the guide catheter 202 may vary. For instance, the gap in cross-sectional diameter between the inner diameter of the guide catheter and the outer diameter of the tube member 220 when expanded may be less than and/or about 0.001 in., 0.002 in., 0.003 in., 0.004 in., or 0.005 in., or any distance therebetween. In specific embodiments, the cross-sectional diameter gap may range from about 0.002 to 0.003 in., or about 0.002 to 0.0035 in. For example, where a guide catheter has an inside diameter of 0.070 in. and the guide extension catheter has an expanded outside diameter of 0.068 in., the gap would be 0.002 in. The diameter gap between an outer diameter of the tube member 220 when expanded and the lumen 204 of the guide catheter 202 may also be generally continuous along a substantial portion of the length or a majority of the length of the tube member 220 in some embodiments, or the diameter gap may increase along one or more distal portions of the tube member 220.

For the portion of the tubular member 220 that has been extended beyond the distal end of the guide catheter 202, and has been released to the uncompressed configuration, the lumen extending through that portion of the tubular member may have an inside dimension, such as a diameter in cross-section and/or an area in cross-section that is larger than an inside diameter and/or the area in cross-section of the lumen 226 of the guide catheter. In some embodiments, the diameter may be approximately equal to the inside diameter of the parent guide catheter.

The length of the tube member 220 can be substantially less than the length of the guide catheter 202. However, the tube member 220 can be designed with any length according to a desired application, such as about 6 to about 45 cm, about 10 to about 35 cm, about 14 to about 25 cm, or about 18 to about 20 cm.

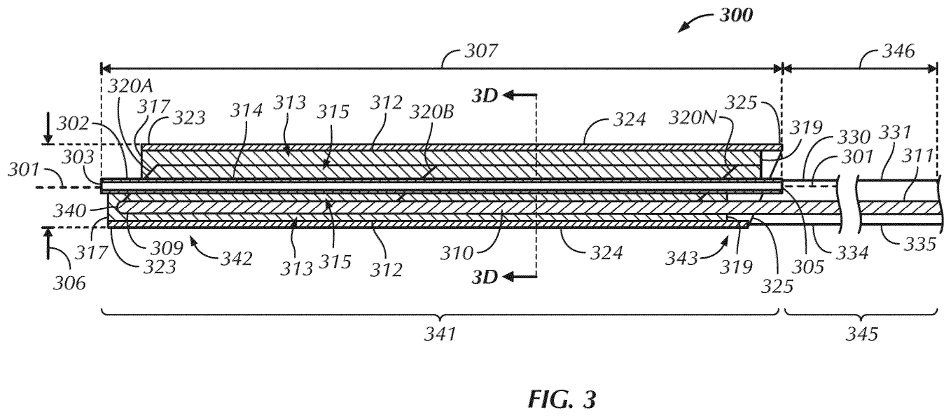

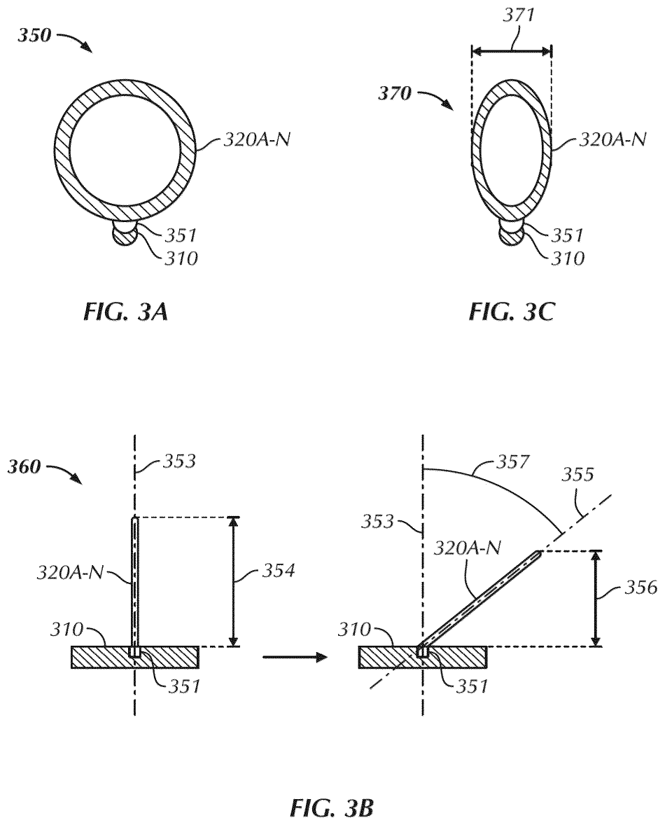

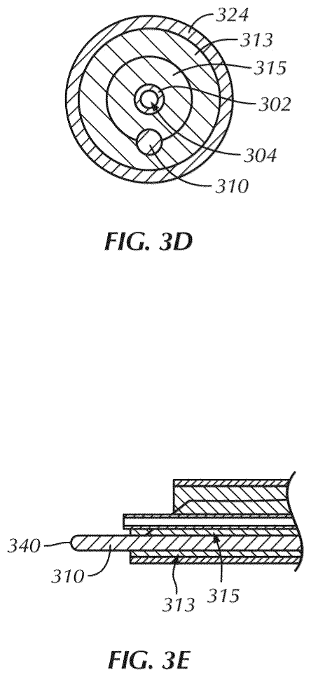

FIG. 3 illustrates a partial cutaway view of a guide extension catheter 300, according to some embodiments. As shown in FIG. 3, guide extension catheter 300 (hereinafter “catheter 300”) includes a tube member 341 extending between a distal end 342 and a proximal end 343 of the tube member. The tube member 341 includes a plurality of components, some of which are arranged to extend in a generally longitudinal direction along a longitudinal axis 301 of the tube member. The plurality of components as illustrated in FIG. 3 include a guidewire support tube 302, an elongate sheath 312, a plurality of coil loops 320A-320N, and an outer delivery cover 324. The relative arrangement of the plurality of components, and various properties, features, and various functions performed by the plurality components included in tube member 341 are further described below.

As shown in FIG. 3, catheter 300 is illustrated in what is referred to as a “compressed configuration.” The compressed configuration is a configuration of catheter 300 that the catheter would be provided in prior to insertion into another catheter, such as a guide catheter. Embodiments of catheter 300 would remain in the compressed configuration as the catheter 300 is being advanced through the guide catheter but has not yet reached a distal end of the guide catheter into which the catheter 300 has been inserted. An overall longitudinal dimension 307 for tube member 341 from distal end 342 to proximal end 343 in various embodiments may vary. As shown in FIG. 3, when in the compressed configuration the tube member 341 has a height dimension 306, as measured in a direction that is perpendicular to longitudinal axis 301 and between the outer surfaces of outer delivery cover 324, which may vary. In various embodiments, the tube member 341 is generally circular shaped in cross-section, and therefore the height dimension 306 corresponds to a diameter of the tube member in cross-section. In various embodiments, tube member 341 does not have a circular shape in cross-section, and may be another shape, such as but not limited to an elliptical shape. In embodiments where the tube member 341 is not approximately circular in shape in cross-section, height dimension 306 corresponds to the dimension that results in the largest cross-sectional dimension of the tube member.

In various embodiments, dimension 307 is measured as extending from a distal end 342 as measured at the distal end of radiopaque maker 340, (which in various embodiments is located as part the lower portion of the tube member 341), to the proximal end 325 of the outer delivery cover 324 of the tube member. As further described below, the overall length dimension 307 of the tube member 341 may vary, for example become smaller in value, when at least a portion of the tube member expands from the compressed configuration to an “uncompressed configuration.” As further described below, when in the uncompressed configuration some portions and/or all portions of the tube member 341 may assume a shape having a height dimension that is larger than the height dimension 306 of the tube member compared to when the tube member is in the compressed configuration.

As further illustrated in FIG. 3, catheter 300 includes an elongate shaft portion 345. Elongate shaft portion 345 extends proximally from the proximal end 343 of the tube member 341, and contains one or more elongate shafts 310, 330, 334. Each of the elongate shafts 310, 330, and 334 are coupled to at least one of the plurality of components included in tube member 341, and wherein the elongate shafts are configured to extend for at least a length 346 that allows a proximal end of each of the elongate shafts to extend beyond a proximal end of a guide catheter (not shown in FIG. 3, but for example guide catheter 202, FIG. 2), when catheter 300 is positioned at the deepest location needed within a vascular of a patient, and thereby allow each of the elongate shaft(s) to be individually manipulated and controlled outside of the patient by a practitioner, such as a physician, when positioned at the deepest location. As illustrated in FIG. 3, elongate shaft 310 terminates proximally at proximal end 311, elongate shaft 330 terminates proximally at proximal end 331, and elongate shaft 334 terminates proximally at proximal end 335.

In some embodiments, each of the elongate shafts 310, 330, and 334 is coupled to a separate one of the components included in the tube member 341. The elongate shafts 310, 330, and 334 are configured to be used to either control the overall positioning of the tube member within and extending partially beyond the distal end of a guide catheter where the tube member 341 is located, or to relocate and/or to completely remove a particular component of the tube member relative to the other components of the tube member and/or completely from the guide catheter where tube member 341 is positioned within. In some embodiments, elongate shaft 310 may also be referred to as a “push member,” and extends proximally from the tube member 341 along the entire length dimension 346 of elongate shaft portion 345, and also extending along the entire length 307 of the tube member 341, having a distal end 309 that extends to or near the distal end 342 of the tube member. Elongate shaft 310 is configured to have flexibility, stiffness, and torqueability qualities that allow forces to be applied to the elongate shaft 310 and that are transferred to the tube member 341 in order to allow the tube member to be advanced through another catheter, such as an outer or guide catheter, and to have the distal end 342 of the tube member extend distally beyond a distal end of the outer or guide catheter, and to retrieve the tube member back out through the proximal end of the outer or guide catheter. In various embodiments, elongate shaft 330 is coupled to a guidewire support tube 302, and is configured to allow a pulling force applied in a proximal direction on the elongate shaft 330 to remove the guidewire support tube from the tube member 341 in a proximal direction, and in various embodiments to allow the guidewire support tube to be completely removed from the outer or guide catheter at some stage of the operation of the guide extension catheter 300 within a vascular of a patient, as further described below. In various embodiments, elongate shaft 334 is coupled to outer delivery cover 324, and is configured to allow a pulling force applied in a proximal direction on the elongate shaft 334 to remove the outer delivery cover 324 from the tube member 341 in a proximal direction, and in various embodiments to allow the outer delivery cover 324 to be completely removed from the outer or guide catheter at some stage of the operation of the guide extension catheter 300 within a vascular of a patient, as further described below. In some embodiments, removal of the outer delivery cover 324 from tube member 341 allows the tube member to be released from the compressed configuration, and to expand to the uncompressed configuration, as further described below.

In various embodiments, elongate shaft 310, acting as the “push member” for tube member 341, may comprise a stainless steel or nitinol core wire and/or a solid core wire wrapped in a smaller wire coil. The elongate shaft 310 may serve as a backbone to the tube member 341, i.e., the elongate shaft 310 may provide column strength and/or bend stiffness to facilitate advancement and withdrawal of the guide extension catheter 300. In some embodiments, a proximal portion of the elongate shaft 310 may include or be surrounded by a removable support member as described in commonly owned U.S. Pat. No. 10,953,197, which is hereby incorporated by reference in its entirety. In various embodiments, neither elongate shaft 330 nor elongate shaft 334 are configured to be used to advance the catheter 300 in a distal direction through an outer or guide catheter. Instead, the elongate shaft 330 may be configured to allow a proximal force exerted on the elongate shaft 330 to be transfer to the guidewire support tube 302 in order to extract the guidewire support tube from the tube member 341 and/or to extract the guidewire support tube from the outer or guide catheter where catheter 300 is position. In addition, the elongate shaft 334 may be configured to allow a proximal force exerted on the elongate shaft 334 to be transferred to the outer delivery cover 324 in order to extract the outer delivery cover from the tube member 341 and/or to extract the outer delivery cover from the outer or guide catheter where catheter 300 is positioned. As such, the elongate shaft 330 and the elongate shaft 334 may be configured using a smaller diameter or small in cross-sectional dimension shaped shaft compared to the same or similar dimensions required for the elongate shaft (“push member”) 310. In various embodiments elongate shaft 330 and elongate shaft 334 may comprise respective stainless steel or nitinol core wires and/or a solid core wire wrapped in a smaller wire coil, having cross-sectional dimensions that may vary.

As further shown in FIG. 3, guidewire support tube 302 extends along longitudinal axis 301 from the distal end 342 to the proximal end 343 of the tube member 341. Guidewire support tube 302 includes a hollow lumen or passageway 304 (FIG. 3D), extending through the entirety of the length of the guidewire support tube 302, having one end of the passageway open at a distal end 303 of the tube 302, and another end of the passageway open at a proximal end 305 of tube 302. The passageway is configured to receive a guidewire (not shown in FIG. 3, but for example guidewire 212, FIG. 2), and allow the guidewire to extend through the passageway of tube 302, thereby allowing the tube 302, along with the tube member 341 that includes the tube 302, to be advanced over the guidewire and at least partially extend into a lumen of an outer or guide catheter. In various embodiments, the shape of the passageway extending through the tube 302 is circular in cross-section. In various embodiments, the shape of the outer surface of the tube 302 is circular cross-section. The passageway through tube 302 may have an inner dimension in cross-section that may vary and may thus accommodate variously sized guidewires. In various embodiments, tube 302 may have an outer dimension that also varies. In various embodiments, the relative inner dimension and outer dimension of the tube 302 provide a wall thickness for the tube 302 that may also vary. The guidewire support tube 302 may be formed of various materials.

As illustrated in FIG. 3, the elongate sheath 312 extends from the distal end 342 to the proximal end 343 of the tube member 341. The elongate sheath 312 forms a generally tubular shape having layers of material in cross-section, including an outer layer 313 and an inner layer 315. A space between the outer layer 313 and the inner layer 315 encloses and encapsulates to portion of elongate shaft 310 that extends along the tube member 341. The outer layer 313 and the inner layer 315 also extend radially to encircle the longitudinal axis 301 of the tube member 341, enclosing and encapsulating the set of coil loops 320A-N. As shown in FIG. 3, each of the coil loops 320A-N is coupled at a point of contact with the elongate shaft 310, and wherein the coil loops extend in a loop shaped ring to surround the longitudinal axis 301 while still being enclosed within the outer layer 313 and the inner layer 315 of the elongate sheath 312. The inner layer 315 of the elongate sheath 312 forms a lumen 314 that extends through the elongate sheath 312 from a distal end 317 to a proximal end 319 of the elongate sheath.

However, because the tube member 341 as shown in FIG. 3 is in a compressed configuration, the lumen 314 is closed off, having the inner layer 315 of the elongate sheath compressed inward radially to encircle and contact the outside surface of guidewire support tube 302. Further, because elongate sheath 312 is configured in the compressed configuration and encapsulates the coil loops 320A-N, each of the coil loops 320A-N is also compressed so that the portion of each coil loop that is adjacent to a point of contact for that loop with elongate shaft 310 is bent inward radially toward the longitudinal axis 301. As shown in FIG. 3, each of the coil loops 320A-N lies in a respective plane that is non-perpendicular to the longitudinal axis 301, such as a plane that is tilted relative to a plane constructed perpendicular to the longitudinal axis. The compression of the coil loops 320A-N allows for the elongate sheath 312 to be compressed around the guidewire support tube 302, thereby closing off the lumen 314 of the elongate sheath, and providing a smaller overall outer dimension 306 for the tube member 341 when in the compressed configuration, while still providing the encapsulation of the coil loops within the layers of material forming the elongate sheath.

In various embodiments, the ring shape of one or more of the coil loops 320A-N may retain a circular shape or loop shape that surrounds the longitudinal axis 301 and encircling the lumen 314. In various embodiments, due to the compression of the coil loops 320A-N, one or more of the coil loops 320A-N may be deformed into non-round shape, such as an elliptical shape, having a major axis that is longer than a minor axis extending across the loop, wherein the major axis may extend from the point of contact of the loop with elongated shaft 310 of a point along the loop that is opposite the point of contact with the elongate shaft 310. The distal end 317 of elongate sheath 312 includes the sealing together of the outer layer 313 and the inner layer 315 of the elongate sheath in order to maintain encapsulation of the coil loops 320A-N, and in some embodiments the encapsulation of a distal end 309 of the elongate shaft 310. In various embodiments, the proximal end 319 of elongate sheath 312 includes the sealing together of the outer layer 313 and the inner layer 315 of the elongate sheath in order to maintain encapsulation of the coil loops 320N, but allowing the elongate shaft 310 to extend proximally away from the seal formed between the outer layer 313 and the inner layer 315. Although described above as individual coil loops, in alternative embodiments the elongate sheath may include a coil or braid and a polymer laminated to the coil or braid that is compressed when the guide extension catheter 300 is in the compressed configuration while providing the urging force that allows the elongate sheath to be expanded when in the uncompressed configuration and provide the lumen or passageway extending through the elongate sheath that is configured to receive and allow interventional treatment devices to pass through the lumen or passageway of the uncompressed guide extension catheter.

In various embodiments, the elongate sheath 312 can include a lubricious layer, a non-crosslinked binder layer, and a crosslinked heat shrink layer. The lubricious layer can include PTFE. The non-crosslinked binder layer can include polyether block amide having a durometer of 35, such as PEBAX 3533 available from Arkema. PEBAX 3533 has a Shore D hardness of 25, a tensile strength at break of 5,660 psi, and a tensile modulus of 2.61-2.76 ksi. The crosslinked heat shrink layer can include polyether block amide having a durometer of 55, such as PEBAX 5533 available from Arkema. PEBAX 5533 has a Shore D hardness of 50, a tensile strength at yield of 1,740 psi, and a tensile modulus of 23.9-24.7 ksi.

In various embodiments, a column strength, a radial strength, and/or a bend stiffness of the elongate sheath 312 may be non-effective, i.e., wherein the radially collapsible properties of the elongate sheath can have no effective column strength, no effective radial strength, and no effective bend stiffness. In other words, any radial force, column force, or bend force can cause the elongate sheath 312 to deflect, collapse, and/or bend, and wherein the elongate sheath 312 will provide no effective resistance to a radial, column, or bend force.

The elongate sheath 312 can have a tensile strength sufficient to prevent tearing of its walls during advancement and withdrawal of an interventional device. For instance, during advancement of an interventional device through the lumen 314 of the elongate sheath 312, the interventional device may provide a force which urges the walls of the elongate sheath 312 radially. The material forming the elongate sheath 312 may have a tensile strength sufficient to withstand the radially outward force of the interventional device such that the interventional device does not tear or otherwise damage the walls of the elongate sheath 312.

The elongate sheath 312 may be durably lubricious on one or both of its inner surface or its outer surface via a hydrophobic, silicone, or polymer coating. The lubricious inner surface of the elongate sheath 312 may be configured to reduce friction between the elongate sheath 312 and the interventional device during insertion and/or withdrawal of the device. The lubricious outer surface of the elongate sheath 312 may be configured to reduce friction between the elongate sheath 312 and the guide catheter during operations, such as advancement and/or withdrawal of the guide extension catheter 300 into and/or from an outer or guide catheter.

The coil loops 320A-N may be formed from a material comprising a stainless steel, nitinol, or another substantially rigid material which possesses a spring-like characteristic that when deformed or twisted tends to urge the coil loops back to a predefined shape. As shown in FIG. 3, each of the coil loops 320 is attached at a respective contact point to elongate shaft 310 and extends away from elongate shaft 310 in a ring or circular shape that encircles the lumen 314, and wherein each of the coil loop is encapsulated between the outer layer 313 and the inner layer 315 of the elongate sheath 312. Because catheter 300 as illustrated in FIG. 3 is in a compressed configuration, each of the coil loops 320A-N is bent or twisted so that the respective loop of each coil lies in a tilted plane relative to a plane that intersects the contact point for that loop and is constructed perpendicular to the longitudinal axis 301. However, due to the spring-like characteristics of the coil loop, each loop provides an urging force that, when catheter 300 is released from the compressed configuration, causes the coil loops to move back toward an orientation wherein the coil loop would lie in a respective perpendicular constructed plane, and thereby expand and open the layers of the elongate sheath 312 so as to open up lumen 314 and provide a passageway extending through the tube member 341. Coil loops 320A-N may be attached to elongate shaft 310 in a manner or using any technique that allows the coil loops to operate in the manner described above and throughout this disclosure, including but not limited to welding, use of adhesive bond, polymer bond, lamination.

As further illustrated in FIG. 3, an outer delivery cover 324 extends from the distal end 342 to the proximal end 343 of the tube member 341. The outer delivery cover 324 comprises a layer of material formed in a tubular shape having an inner surface located adjacent to and in contact with the outer layer 313 of the elongate sheath 312 and extending from a distal end 323 to a proximal end 325 of the outer delivery cover. The outer delivery cover 324 encircles the longitudinal axis 301, the guidewire support tube 302, one or more of coil loops 320A-N, and the elongate sheath 312 along all or almost all of the length 307 of the tube member. The outer delivery cover 324 is configured to maintain the elongate sheath 312 and the coil loops 320A-N in the compressed configuration as shown in FIG. 3 due to the size and shape of the outer delivery cover 324. An inside diameter in cross-section of the outer delivery cover 324 is configured so that the elongate sheath 312 and the coil loops 320A-N will be compressed into contact with the guidewire support tube 302 when the outer delivery cover 324 is positioned around the tube member 341. The outer delivery cover 324 is formed from a material that while flexible in a longitudinal direction, is configured to not expand radially away from the longitudinal axis 301 despite outward forces exerted on the outer delivery cover 324 due to the compression of the elongate sheath 312 and/or by the coil loops 320A-N. In various embodiments, the outer delivery cover 324 comprises a material formed from a polymer such as but not limited to PTFE, ePTFE, nylon, PET.

Outer delivery cover 324 is configured to be removable from the tube member 341 at some stage of the operation of the guide extension catheter in order to allow the coil members 320A-N and the elongate sheath 312 to expand from the compressed configuration to the uncompressed configuration, including opening up a passageway extending through lumen 314 of the elongate sheath 312, and thereby allowing for the introduction of one or more treatment devices to pass through and beyond the distal end 317 of the elongate sheath for use during a treatment procedure being performed within the vasculature of a patient where the guide extension catheter 300 has been deployed. In various embodiments, the removal of the outer delivery cover 324 is accomplished by applying a force proximally on elongate shaft 334 that is coupled to the outer delivery cover 324, wherein the proximally applied force is transferred to the outer delivery cover 324 to slide the outer delivery cover 324 over and away from the elongate sheath 312 in a proximal direction until the outer delivery cover 324 no longer extends over the tube member 341. In various embodiments, the outer delivery cover 324 is formed from a lubricious material that helps enable the movement of the outer delivery cover 324 relative to the outer layer 313 of the elongate sheath 312. In various embodiments, outer delivery cover 324 may incorporate a perforation or weakened portion of the material forming the outer delivery cover that is configured to rupture or tear, for example along the longitudinal axis of the outer delivery cover when the force is applied to elongate shaft 334, the rupture or tear configured to allow the outer delivery cover 324 to more easily be moved proximally relative to the outer layer 313 of the elongate sheath 312.

Once the outer delivery cover 324 has been removed from the tube member 341, the urgent forces provided by coil loops 320A-N will cause the coil loops to extend to a less compressed angle relative to the longitudinal axis 301, which in turn will expand the elongate sheath 312 so that the lumen 314 extending through the elongate sheath will open to form a passageway extending through the elongate sheath.

FIGS. 3A-3C illustrate views 350, 360, and 370 of configurations of the coil loops 320A-N as included in a guide extension catheter of FIG. 3, according to some embodiments.

FIG. 3A illustrates a front view 350 of a coil loop (any one of coil loops 320A-N, FIG. 3) attached to the elongate shaft 310 at a bonded area 351, the view taken looking along the longitudinal axis of elongate shaft 310. The coil loop may be formed of a wire or other circular shaped element that is formed into a loop or ring shape as shown in view 350. The coil loop may comprise a material, such as nitinol or stainless steel, that has spring properties. The coil is bonded at one place around the perimeter of the loop or ring shape at bonding area 351. Bonding area 351 may form a bond between the coil loop and elongate shaft 310 that fixes the coil loop at one position longitudinally along the elongate shaft, but that allows the remaining portion of the coil loop to be compressed or to bend in various directions relative to the elongate shaft 310 when the coil loop is held in these various positions by some other component(s) of the tube member of the guide extension catheter where the coil loop are located. The spring properties of the coil loop will urge the coil loop to return to the shape and configuration as shown in view 350 when no longer being forcibly retained in the bent or otherwise compressed configuration.

FIG. 3B illustrates a pair of side views 360 of any one of coil loops 320A-N, (FIG. 3), attached to the elongate shaft 310 at a bonded area 351. In the left-hand side of FIG. 3B, coil loop is shown in an unbent configuration, wherein a plane 353 that the coil loop lies in is perpendicular to the longitudinal axis of the elongate shaft 310 at the bonding area 351. The maximum overall height of the coil loop is illustrated by dimension line 354. In the right-hand side of FIG. 3B, the coil loop is shown in a bent-over configuration, wherein a plane 355 that the coil loop lies in is not perpendicular to the longitudinal axis of the elongate shaft 310, and extends through the bonding area 351 at some acute angle 357 relative to the perpendicular plane 353. The maximum overall height of the coil loop in this bent-over configuration is illustrated by dimension line 356, which is smaller in value than the value for dimension 354. When in the configuration as shown in the right-hand side of view 360, an overall cross-sectional dimension for the tube member where the coil loop is located may be smaller than when the tube member is allowed to return to an expanded configuration that includes the coil loops extended to a configuration the same as or closer to the configuration illustrated in the left-hand portion of view 360.

FIG. 3C illustrates a front view 370 of a coil loop (any one of coil loops 320A-N, FIG. 3) attached to the elongate shaft 310 at a bonded area 351, the view taken looking along the longitudinal axis of elongate shaft 310. In view 370, the coil loop is both bent over in a manner as described in the right-hand side of view 360 (FIG. 3B), but is also compressed laterally in a direction perpendicular to the longitudinal axis of elongate shaft 310 across a width axis of the coil loop. When in the bent-over and compressed configuration as shown in view 370, the coil loop may take on an elliptical shape in the plane that the coil loop lies in, resulting in a smaller dimensional value for the width axis 371 across the cool loop along the center axis. When in the configuration as shown in view 370, an overall cross-sectional dimension for the tube member where the coil loop is located may be smaller than when the tube member is allowed to return to an expanded configuration that includes the coil loops extended to a configuration the same as or closer to the configuration illustrated in the left-hand portion of view 360.

FIG. 3D illustrates a cross-sectional view of a portion of the of the guide extension catheter 300 of FIG. 3, according to some embodiments. The cross-sectional view is taken along dashed line 3D-3D of guide extension catheter 300 in FIG. 3, looking toward the distal end of the guide extension catheter. As shown in FIG. 3D, the guidewire support tube 302 extends along the longitudinal axis 301 of the guide extension catheter and encircles a lumen 304 that extends throughout the length of the guidewire support tube longitudinally. An inner layer 315 of the elongate sheath 312 encircles the guidewire support tube 302, wherein the inner surface of the inner layer 315 may contact some portion radially, or all portions radially, of an outer surface of the guidewire support tube. An outer layer 313 of the elongate sheath 312 encircles the inner layer 315, wherein the inner surface of the outer layer 313 may contact some portion radially, or all portions radially, of an outer surface of inner layer 315. The outer delivery cover 324 encircles the outer layer 313, wherein the inner surface of the outer delivery cover 324 may contact some portion radially, or all portions radially, of an outer surface of outer layer 313. As described above, the outer delivery cover 324 is configured to maintain the guide extension catheter 300 in the compressed configuration, as shown in FIG. 3 and in FIG. 3D, until the outer delivery cover is removed from a position encircling the outer layer 313.

FIG. 3E illustrates an end portion of the guide extension catheter 300 of FIG. 3, according to some embodiments. As shown in FIG. 3E, instead of elongate shaft 310 having a distal end that is encapsulated within the elongate sheath 312 as shown in FIG. 3, in FIG. 3E the elongate shaft 310 extends out of and distally of the distal end portion of the elongate sheath 312 formed by the outer layer 313 and the inner layer 315. As illustrated in FIG. 3E, the distal end of elongate shaft 310 may be capped in a tip member 340, which may be formed of a soft or flexible material, and/or which may incorporate or be comprised of a radiopaque material, which may be used to locate the position of the distal end of the guide extension catheter 300 when positioned within the vasculature of a patient. A distal end of the elongate shaft 310 may include an atraumatic guidewire-like distal tip. In some embodiments, the atraumatic guidewire-like distal tip includes a tapered core surrounded by a coil, and in some embodiments, the atraumatic guidewire-like distal tip includes a steerable tip.

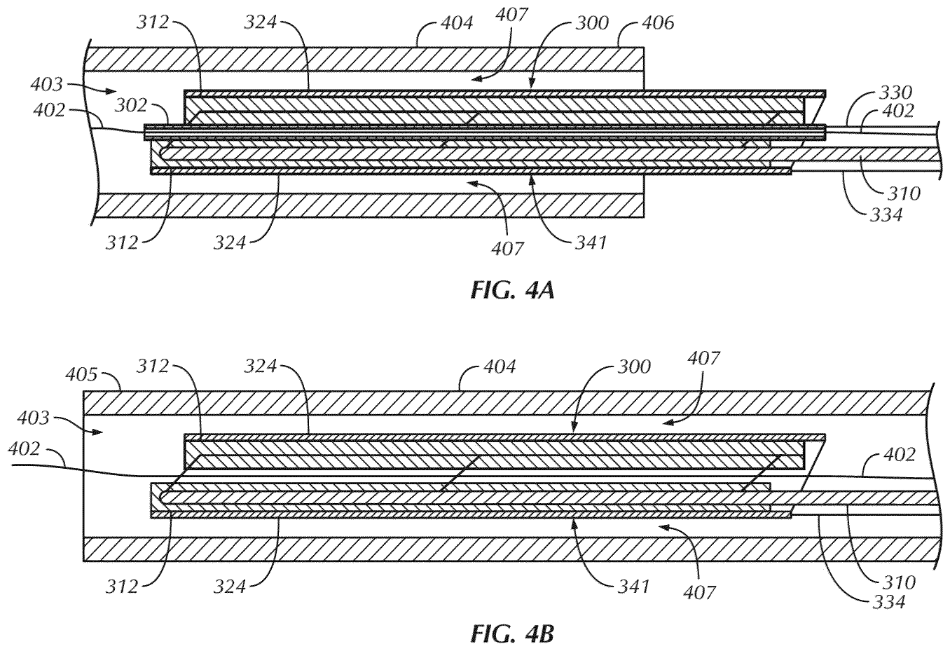

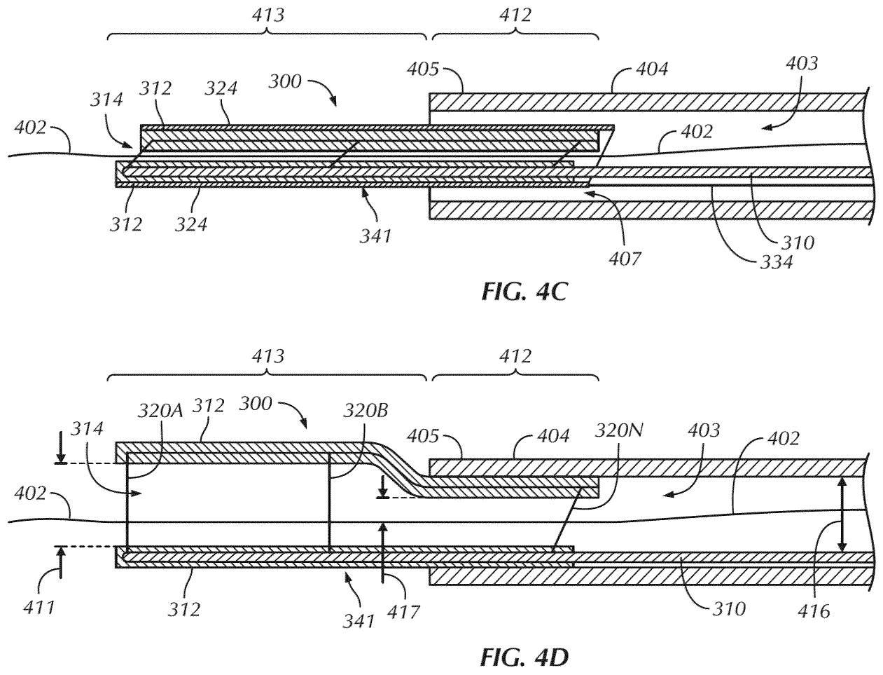

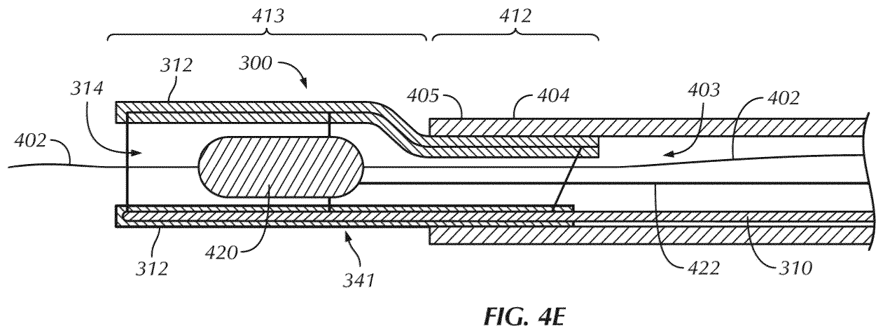

FIGS. 4A-4E illustrate various stages of deployment of the guide extension catheter 300 of FIG. 3, according to various embodiments. FIG. 4A is a cutaway view showing guide extension catheter 300 positioned at least partially within a lumen 403 of a guide catheter 404. In various embodiments, guide catheter 404 is positioned within the vasculature of a patient (not shown in FIG. 4A-4E, but for example coronary artery 210, FIG. 2), wherein the distal end 405 of the guide catheter 404 may be positioned adjacent to an ostium of the coronary artery. Catheter 300 may be initially advanced at least partially into lumen 403 at the proximal end 406 of guide catheter 404 by feeding the guidewire support tube 302 over guidewire 402, wherein guidewire 402 extends through the entirety of the length of the guide catheter 404. After removal of the guidewire support tube 302, the guide extension catheter 300 may be further extended through the lumen 403 of guide catheter 404 until some distal portion of the guide extension catheter extends distally beyond the distal end 405 of the guide catheter, as illustrated and further described below with respect to FIGS. 4B-4E.

As shown in FIG. 4A, catheter 300 is configured in the compressed configuration as described above, wherein the elongate sheath 312 is maintained in the compressed configuration by the presence of the outer delivery cover 324. Depending on the relative inside diameter of lumen 403 of the guide catheter 404 and the overall diameter of catheter 300 in cross-section, there may be some amount of space 407 between the outer delivery cover 324 and the inner surface of the lumen 403 of the guide catheter 404. As shown in FIG. 4A, each of the elongate shaft (push member) 310, the optionally included elongate shaft 330 coupled to the guidewire support tube 302, and the elongate shaft 334 coupled to the outer delivery cover 324, are present and extend proximally from the tube member 341 of catheter 300 to and beyond the proximal end 406 of the guide catheter 404. Guide extension catheter 300 may be at least partially advanced into the lumen 403 of the guide catheter 404 to the position shown in FIG. 4A by feeding the guidewire support tube 302 over the guidewire 402 and advancing the guide extension catheter into the lumen 403 of the guide catheter until at least some portion or all of the guide extension catheter is positioned within lumen 403. Once positioned as show in FIG. 4A, the guidewire support tube 302 may be removed from the guide extension catheter 300, in some embodiments by exerting a force in a proximal direction on the guidewire support tube itself, and/or by exerting a force in a proximal direction on a device, such as elongate shaft 330, which is coupled to the guidewire support tube. With the guidewire support tube removed, the guide extension catheter 300 may be further advanced through the guide catheter 404 to a location near the distal end 405 of the guide catheter by applying a force exerted in a distal direction on the elongate shaft 310 to cause tube member 341 to be advanced from a proximal end 406 of guide catheter 404 to the position within the guide catheter as shown in FIG. 4B, in various embodiments by feeding the guidewire support tube 302 over the guidewire 402 as the tube member 341 is advanced toward the distal end 405 of the guide catheter. As further illustrated and described with respect to FIG. 4B, when tube member 341 is positioned near the distal end 405 of guide catheter 404, the guidewire 402 may extend distally beyond both the tube member 341 and beyond the distal end 405 of the guide catheter 404.

FIG. 4B is a cutaway view showing guide extension catheter 300 positioned within the lumen 403 of the guide catheter 404 as described above with respect to FIG. 4A. As shown in FIG. 4B, catheter 300 is still in the compressed configuration, is still positioned completely with lumen 403 of the guide catheter 404, and having guidewire 402 extending through the lumen 314 of the elongate sheath 312 and beyond the distal end 405 of the guide catheter.

FIG. 4C is a cutaway view showing guide extension catheter 300 partially extending distally beyond the distal end 405 of guide catheter 404 in accordance with various embodiments. The positioning of guide extension catheter 300 as shown in FIG. 4C may be achieved from the positioning of the guide extension catheter 300 as shown in FIG. 4B by exertion of a force in a distal direction on push member 310, for example from a position of the push member 310 extending out of the proximal end of the guide catheter 404, for example by a physician. When positioned as shown in FIG. 4C, a proximal portion 412 of the tube member 341 remains located within the lumen 403 of the guide catheter 404, while a distal portion 413 of the tube member 341 extends distally beyond the distal end 405 of the guide catheter, with the guidewire 402 extending through the entirety of lumen 314, and extending distally beyond the distal portion 413 of the tube member. As shown in FIG. 4C, all portions of the elongate sheath 312, including the portion of the elongate sheath included in the proximal portion 412 and in the distal portion 413 of the tube member 341, remain in the compressed configuration due to the presence of the outer delivery cover 324 remaining present and encircling the outer surfaces of the elongate sheath 312. Elongate shafts 310 and 334 remain coupled to one or more components of the tube member 341 and extend proximally within the lumen 403 of the guide catheter 404, the elongate shafts having respective proximal ends that extend out of and beyond a proximal end of the guide catheter.

FIG. 4D is a cutaway view showing guide extension catheter 300 in an expanded configuration, according to some embodiments. The positioning of guide extension catheter 300 as shown in FIG. 4D relative to guide catheter 404 is the same as illustrated in FIG. 4C, with the proximal portion 412 of the tube member 341 remaining located within the lumen 403 of the guide catheter 404, while the distal portion 413 of the tube member 341 extends distally beyond the distal end 405 of the guide catheter 404. Catheter 300 has been expanded from the compressed configuration as shown in FIG. 4C to the uncompressed configuration as shown in FIG. 4D.

When positioned as shown in FIG. 4D, a proximal portion 412 of the guide extension catheter 300 remains located within the lumen 403 of the guide catheter 404, while a distal portion of the guide extension catheter 300 extends distally beyond the distal end 405 of the guide catheter, with the guidewire 402 extending through the entirety and distally beyond the distal portion 413 of the guide extension catheter. Guidewire 402 remains extending through the entirety of lumen 314 and extending distally beyond the distal portion 413 of the tube member 341.

A difference between the configuration of guide extension catheter 300 as shown in FIG. 4C and the configuration of the guide extension catheter as shown in FIG. 4D is that as illustrated in FIG. 4D, the outer delivery layer 324 has been removed from its position encircling the elongate sheath 312, which thereby allows the coil loops 320A-N to urge and expand the elongate sheath from the compressed configuration to an expanded configuration, and in the process opening up the lumen 314 extending through the tube member 341, and forming a passageway encircled by elongate sheath 312 and the coil loops 320A-N from the distal to the proximal ends of the tubular member.

In various embodiments, the outer delivery cover 324 may be removed by applying a pulling force in a proximal direction on elongate shaft 334 (FIG. 4C), thereby causing the outer delivery cover 324 to be extracted from encircling the tube member 341. In various embodiments, the outer delivery cover 324 is completely removed from the lumen 403 of the guide catheter 404 by pulling elongate shaft 334 proximally until the outer delivery cover 324 is extracted through the proximal end of the guide catheter 404. Elongate shaft 310 remains coupled to one or more components of the tube member 341 and extends proximally within the lumen 403 of the guide catheter 404, the elongate shaft 310 having a proximal end that extends out of and beyond a proximal end of the guide catheter. The extension of the elongate shaft 310 out and beyond the proximal end of the guide catheter 404 allows an operator, such as a physician, to control and maintain the position of the tube member 341 and the catheter 300 during various procedures, such as the removal of the outer delivery cover 324 from the tube member, during procedures involving the advancement and retraction of one or more treatment devices through the catheter 300, and including the retrieval of the catheter 300 from the guide catheter 404 once the procedure(s) being performed on the patient have been completed.

In various embodiments, as shown in FIG. 4D, the proximal portion 412 of the guide extension catheter 300 that remains positioned within the lumen 403 of the guide catheter 404 may be expanded so that the outside surfaces of the elongate sheath 312 are in contact, around at least some or all of the perimeter of these outer surfaces, with the inner surface(s) forming the lumen 403 within the guide catheter 404. In various embodiments, and as also shown in FIG. 4D, the distal portion 413 of the tube member 341 that remain positioned beyond the distal end 405 of the guide catheter 404 may be in an expanded configuration. In various embodiments, when in this expanded configuration the outside surfaces of the elongate sheath in the distal portion 413 may be in contact with, around at least some or all of the perimeter of these outer surfaces, the inner surface(s) of a vascular of a patient where the distal portion 413 of the tube member 341 is located, including being in contact with a vascular that is located distally beyond the ostium of the patient. In various embodiments, a value for an internal dimension, such as a diameter 411 in cross-section of the lumen 314 of the distal portion 413 tube member 341 when in the expanded configuration as shown in FIG. 4D is larger than an internal dimension, such as a diameter 416 in cross-section of the lumen 403 of the guide catheter 404.

In various embodiments, when in the expanded configuration one or more of the loops 320A-N that are positioned in the distal portion 413 may be extended to lie in a plane that is perpendicular or nearly perpendicular to the line of axis of the push member 310, which may provide a maximum dimension 411 in cross-section available for the portion of lumen 314 that is located in the distal portion 413. In various embodiments, when in the expanded configuration one or more of the loops 320A-N that are positioned in the proximal portion 412 may remain partially radially compressed in a direction toward the axis of push member 310 so that these loops lie a plane that is tilted an angle less than ninety degrees relative to the line of axis of the push member 310. As a result, a value for an internal dimension, such as a diameter 417 in cross-section for the portion of the lumen 314 positioned within proximal portion 412 when the guide extension catheter 300 is in the expanded configuration as shown in FIG. 4D, may be smaller than the internal dimension 416 for the lumen 403 of the guide catheter 404. In various embodiments, a diameter 416 of the lumen 403 of the guide catheter 404 near distal end 405 may vary, a diameter 417 of the portion of the lumen 314 of the guide extension catheter 300 that is located within the proximal portion 412 may also vary, and a diameter 411 of the portion of lumen 314 of the guide extension catheter 300 that is located within the distal portion 413 may also vary.

FIG. 4E illustrates a partial cutaway view of the guide extension catheter 300 of FIG. 3 deployed in a guide catheter 404 and receiving a treatment device 420, according to various embodiments. Guide extension catheter 300 as illustrated in FIG. 4E is in the expanded configuration, and in the same or approximately the same position as was illustrated and described above with respect to FIG. 4D. As shown in FIG. 4E, a treatment device 420 coupled to a treatment device push member 422 has been advanced through the lumen 403 of the guide catheter 404, through the lumen 314 of the tube member 341 that is located within the proximal portion 412, and into position within the distal portion 413 of lumen 314 that is located distally of the distal end 405 of the guide catheter 404. Guidewire 402 remains extending distally from lumen 314. As would be understood by one of ordinary skill in the art, the treatment device 420 may be further advanced out of the distal end of the tube member 341 and on to a treatment site within a vascular of a patient (not shown in FIG. 4E, but for example stenotic lesion 218, FIG. 2), in order to provide treatment or other procedures at the treatment site. Because the distal portion 413 of the tube member 341 may extend further into a vascular than would be possible or practical for the distal end 405 of the guide catheter 404, the delivery of the treatment device when exiting the guide extension catheter 300 may be closer to the location of the treatment site, thus reducing the chances of damage to the vascular wall proximal to the treatment site potentially caused by the advancement of the treatment device done in the absence of the guide extension catheter.

Further, the positioning of the distal portion 413 of the tube member 341 of guide extension catheter being extended to a further location distally within the vascular provides additional stability for the distal end 405 of the guide catheter 404, thereby lessening the possibility of dislodging the distal end 405 of the guide catheter from the vascular structure, such as the ostium, during the time the treatment device 420 is being advanced toward the treatment site and/or during the treatment and/or the procedure(s) being performed on or at the treatment site. In addition, because the coil loops 320A-N remain encapsulated within the layers of the elongate sheath 312 when catheter 300 is in the expanded configuration, the possibility of the treatment device 420 and or the push member 310 getting caught on any of the coil loops 320A-N is reduced or completely eliminated. Upon completion of any treatment or other procedures utilizing the treatment device 420, the treatment device may be retrieved back into the lumen 314 utilizing a proximally exerted pulling force applied to push member 422, wherein the treatment device 420 may be fully extracted out the proximal end of guide catheter 404. Upon completion of any treatment or other procedures utilizing the treatment device 420 and removal of the treatment device from the guide catheter 404, the tube member 341 may also be retrieved back through lumen 403 of the guide catheter 404 utilizing a proximally exerted pulling force applied to elongate shaft 310, wherein the treatment device tube member 341 may be fully extracted out the proximal end of guide catheter 404 utilizing elongate shaft 310 for the retrieval.

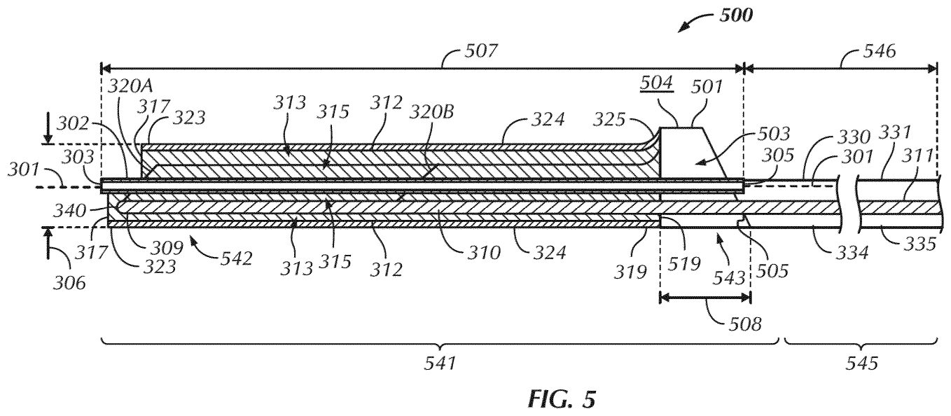

FIG. 5 illustrates a partial cutaway view of a guide extension catheter 500, according to some embodiments. Guide extension catheter 500 includes many of the same or similar components, arranged in a same or similar manner, as described above with respect to guide extension catheter 300 and as shown illustrated in FIG. 3. As such, these same or similar components as included in guide extension catheter 500 retain a corresponding three-hundred range reference number in the illustration and description related to FIG. 5. These same or similar components may be configured to provide the same or similar features and to perform the same or similar functions as their corresponding components of guide extension catheter 300. The differences in guide extension catheter 500 are further described below.

Guide extension catheter 500 includes a tube member 541, with an elongate shaft portion 545 coupled to and extending proximally from the tube member. Tube member 541 includes a guidewire support tube 302, an elongate sheath 312, and an outer delivery cover 324 arranged around a longitudinal axis 301 of the tube element configured in a manner similar to that of guide extension catheter 300. In addition, guide extension catheter 500 differs from guide extension catheter 300 in at least that guide extension catheter 500 includes tube member proximal end cap (proximal cap) 501.

In various embodiments, proximal cap 501 may be formed from a rigid material having a fixed shape and structure and may be positioned at the proximal end of the tube member. In various embodiments, proximal cap 501 may be formed from a hard plastic material. Wherein tube member 541 of the guide catheter has a length dimension 507 extending along the longitudinal axis, a length dimension 508 of the proximal cap along the longitudinal axis 301 may vary. Elongate shafts 310, 330, 334 of elongate shaft portion 545 are configured to extend for at least a length 546 that allows a proximal end of each of the elongate shafts to extend beyond a proximal end of a guide catheter when catheter 500 is positioned at the deepest location needed within a vascular of a patient, thereby allowing each of the elongate shaft(s) to be individually manipulated and controlled outside of the patient by a practitioner, such as a physician, when positioned at the deepest location.

Proximal cap 501 includes an outer surface 504, which may be circular in cross-section, and sized to fit within the lumen of a guide catheter where the guide extension catheter 500 is intended to be advanced through and positioned within. Proximal cap 501 further includes a lumen 503 forming a passageway extending from a proximal face 505 to a distal end 519 of the proximal cap. The interior shape of the lumen 503 in various embodiments is circular in cross- section and has an interior diameter in cross-section that may vary. In various embodiments, the proximal end 325 of the outer delivery cover 324 and the proximal end 319 of the elongate sheath 312 are bonded to the distal end 519 of the proximal cap 501. One or more coil loops 320 A-B may be encapsulated within the elongate sheath 312 in a same or similar manner as described above with respect to coil loops 320A-N. As shown in FIG. 5, elongate shaft (push member) 310 may extend longitudinally through the proximal cap from the proximal face 505 to and distally beyond the distal end 519, and to or nearly to the distal end of the tube member 541. The elongate shaft 310 is coupled to the tube member and/or to the proximal cap 501 in order to allow forces applied to the elongate shaft 310 to control the movements, both longitudinally and rotationally, of the tube member 541. Although described above as individual coil loops, in alternative embodiments the elongate sheath includes a coil or braid and a polymer laminated to the coil or braid that is compressed when the guide extension catheter 500 is in the compressed configuration while providing the urging force that allows the elongate sheath to be expanded when in the uncompressed configuration and provide the lumen or passageway extending through the elongate sheath that is configured to receive and allow interventional treatment devices to pass through the lumen or passageway of the uncompressed guide extension catheter.

In various embodiments, proximal face 505 of the proximal cap 501 may include a concave opening leading into the lumen 503. The concave opening may be configured to ease the insertion of a guidewire (not shown) or an interventional device (not shown) into the lumen 503. For instance, the concave opening may provide a larger area to receive an interventional device into the tube member than an area associated with an opening oriented perpendicular to the longitudinal axis of the tube member. In some embodiments, the proximal cap 501 may include an extended concave track defining a half-pipe feature configured to help guide an interventional device into the lumen 503 extending through the proximal cap 501. Exemplary embodiments of half-pipes, concave tracks, and other first reinforcement members and features are described in commonly owned U.S. Pat. Pub. No. 2019/0247619, which is hereby incorporated by reference in its entirety.

The proximal cap 501 can have a length 508 defined between the proximal face 505 and the distal end 519. The proximal cap 501 can have an inner diameter dᵢ (also referred to as a first lumen diameter), an outer diameter dₒ, and a wall thickness. In some embodiments, the length 508 of the proximal cap 501 may be as small as dᵢ/10.

In some embodiments, proximal cap 501 can be formed from an inner polymer layer, an outer polymer layer, and/or a reinforcement layer (e.g., braid or coil) disposed between or adjacent to the polymer layers. According to such examples, the inner polymer layer can be composed of, or coated with, silicone, PTFE or another lubricious material to provide a slippery surface for received interventional devices. The outer polymer layer can include one or more materials, such as polyurethane, polyethylene, polyolefin, or polyether block amide of sequentially diminishing durometers along the tube member's length, and it can be coated with a friction-reducing material (e.g., a hydrophilic material) to facilitate insertion and trackability through vasculature and a guide catheter. The reinforcing braid or coil, in embodiments featuring a braid or coil, can be formed of stainless steel or a platinum alloy, for example, and can extend between the polymer layers along at least a portion of the tube member's length.

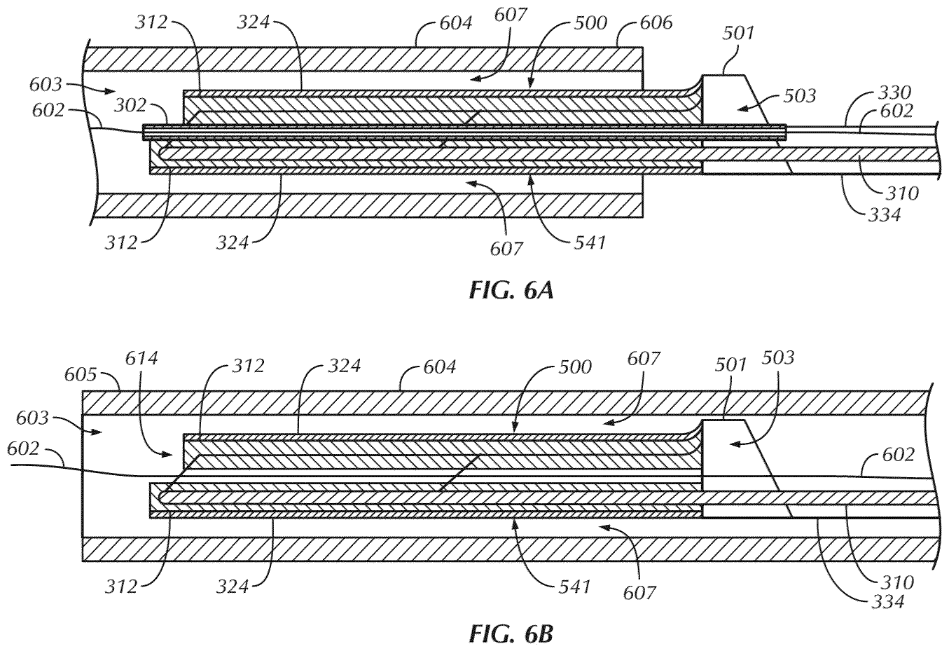

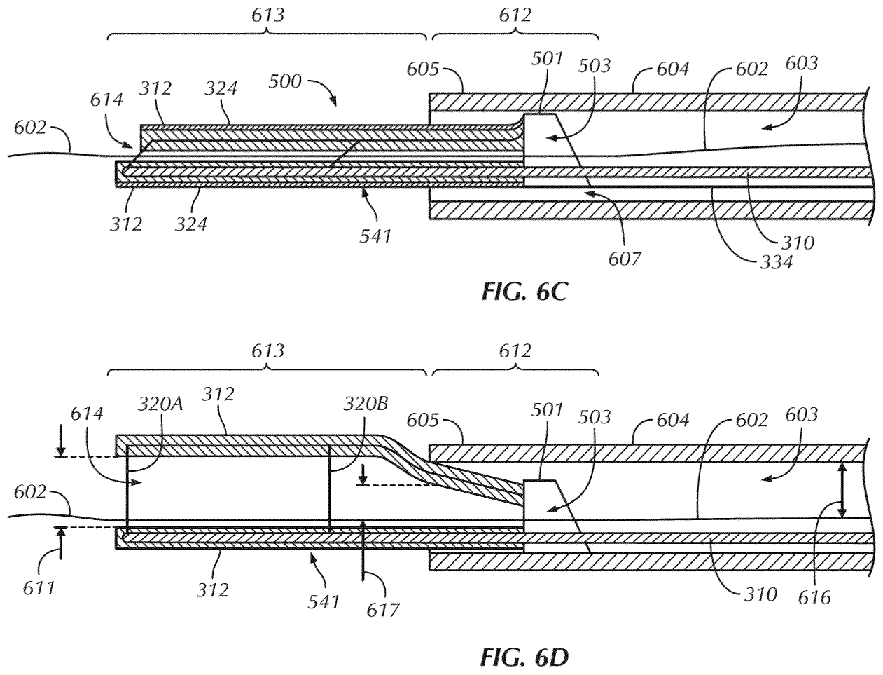

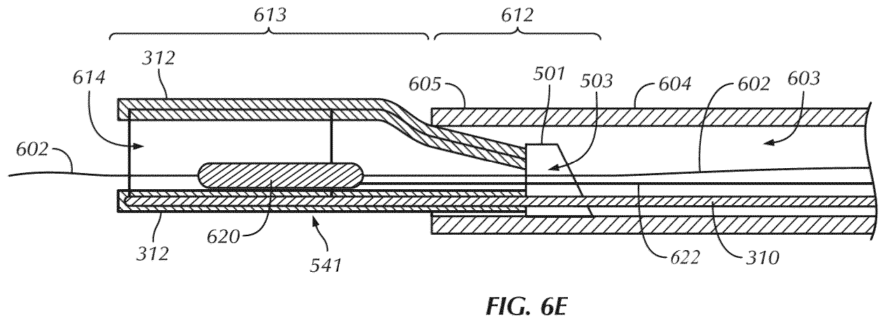

FIGS. 6A-6E illustrate various stages of deployment of the guide extension catheter 500 of FIG. 5, according to various embodiments. FIG. 6A is a cutaway view showing guide extension catheter 500 at least partially positioned within a lumen 603 of a guide catheter 604. In various embodiments, guide catheter 604 is positioned within a vascular of a patient (not shown in FIG. 6A-6E, but for example coronary artery 210, FIG. 2), wherein the distal end 605 of the guide catheter 604 may be positioned adjacent to an ostium of the coronary artery. Catheter 500 may be initially advanced at least partially into the lumen 603 at the proximal end 606 of guide catheter 604 by feeding the guidewire support tube 302 over guidewire 602, wherein guidewire 602 extends through the entirety of the length of the guide catheter 604. After removal of the guidewire support tube 302, the guide extension catheter 500 may be further extended into and through lumen 603 of the guide catheter 604 until some distal portion of the guide extension catheter extends distally beyond the distal end 605 of the guide catheter, as illustrated and further described below with respect to FIGS. 6B-6E. It is noted that FIGS. 6A-6E generally correspond to the same stages of deployment using guide extension catheter 500 as is depicted in FIGS. 4A-4E, but using guide extension catheter 500. A difference between guide extension catheter 300 and guide extension catheter 500 includes that guide extension catheter 500 includes a proximal cap 501 as part of the tube member, wherein guide extension catheter 300 does not include a proximal cap comprising a rigid structure as part of the tube member.

As shown in FIG. 6A, guide extension catheter 500 is configured in the compressed configuration as described above with respect to FIG. 5, wherein the elongate sheath 312 is maintained in the compressed configuration by the presence of the outer delivery cover 324, and wherein the elongate sheath is bonded to the distal end 519 of the proximal cap 501. Depending on the relative inside diameter of the guide catheter 604 and the overall diameter of guide extension catheter 500 in cross-section, there may be some amount of space 607 between the outer delivery cover 324 and the inner surface of the lumen 603 of the guide catheter 604. As shown in FIG. 6A, each of the elongate shaft 310, the optionally included elongate shaft 330 coupled to the guidewire support tube 302, and the elongate shaft 334 coupled to the outer delivery cover 324 are present and extend proximally from the tube member 541 of catheter 500 to and beyond a proximal end 606 of the guide catheter 604. Guide extension catheter 500 may be at least partially advanced into the lumen 603 of the guide catheter 604 to the position shown in FIG. 6A by feeding the guidewire support tube 302 over the guidewire 602 and advancing the guide extension catheter into the lumen 603 of the guide catheter until at least some portion or all of the guide extension catheter is positioned within lumen 603. Once positioned as show in FIG. 6A, the guidewire support tube 302 may be removed from the guide extension catheter 500, in some embodiments by exerting a force in a proximal direction on the guidewire support tube itself, and/or by exerting a force in a proximal direction on a device, such as elongate shaft 330, which is coupled to the guidewire support tube. With the guidewire support tube removed, the guide extension catheter 500 may be further advanced through the guide catheter 604 to a location near the distal end 605 of the guide catheter 604 by applying a force exerted in a distal direction on the elongate shaft 310 to cause tube member 541 to be advanced from a proximal end 606 of guide catheter 604 to the position within the guide catheter as shown in FIG. 6B, in various embodiments by feeding the guidewire support tube 302 over the guidewire 602 as the tube member 541 is advanced toward the distal end 605 of the guide catheter. As further illustrated and described below with respect to FIG. 6B, when tube member 541 is positioned near the distal end 605 of guide catheter 604, the guidewire 602 may extend distally beyond both the tube member 541 and beyond the distal end 605 of the guide catheter 604.

FIG. 6B is a cutaway view showing guide extension catheter 500 positioned within the lumen 603 of the guide catheter 604 as described above with respect to FIG. 6A, but now having the guidewire support tube 302 removed from the tube member portion of the guide extension catheter 500. As shown in FIG. 6B, catheter 500 is still in the compressed configuration, is still positioned completely with lumen 603 of the guide catheter 604, and having guidewire 602 extending through the lumen 614 of the elongate sheath 312 and beyond the distal end 605 of the guide catheter.

FIG. 6C is a cutaway view showing a portion of the tube member 541 of the guide extension catheter 500 partially extending distally beyond the distal end 605 of guide catheter 604, in accordance with various embodiments. The positioning of guide extension catheter 500 as shown in FIG. 6C may be achieved from the positioning of the guide extension catheter 500 as shown in FIG. 6B by exertion of a force in a distal direction on push member 310, for example from a position of the push member extending out of the proximal end of the guide catheter 604, for example by a physician. When positioned as shown in FIG. 6C, a proximal portion 612 of the guide extension catheter 500, including the entirety of proximal cap 501, remains located within the lumen 603 of the guide catheter 604, while a distal portion 613 of the guide extension catheter 500 extends distally beyond the distal end 605 of the guide catheter, with the guidewire 602 extending through the entirety and distally beyond the distal portion 613 of the guide extension catheter. As shown in FIG. 6C, all portions of the elongate sheath 312, including the portion of the elongate sheath included in the proximal portion 612 and in the distal portion 613 of the tube member 541, remain in the compressed configuration due to the presence of the outer delivery cover 324 remaining present and encircling the outer surfaces of the elongate sheath. Elongate shafts 310 and 334 remain coupled to one or more components of the tube member 541 and extend proximally within the lumen 603 of the guide catheter 604, the elongate shafts having respective proximal ends that extend out of and beyond a proximal end of the guide catheter.

FIG. 6D is a cutaway view showing guide extension catheter 500 in an expanded configuration, according to some embodiments. The positioning of guide extension catheter 500 as shown in FIG. 6D relative to guide catheter 604 is the same as illustrated in FIG. 6C, with the proximal portion 612 of the tube member 541, as well as proximal cap 501, remaining located within the lumen 603 of the guide catheter 604, while the distal portion 613 of the tube member 541 extends distally beyond the distal end 605 of the guide catheter 604. Catheter 500 has been expanded from the compressed configuration as shown in FIG. 6C to the uncompressed configuration as shown in FIG. 6D.

When positioned as shown in FIG. 6D, a proximal portion 612 of the guide extension catheter 500 remains located within the lumen 603 of the guide catheter 604, while a distal portion 613 of the guide extension catheter 500 extends distally beyond the distal end 605 of the guide catheter, with the guidewire 602 extending through the entirety and distally beyond the distal portion 613 of the guide extension catheter. Guidewire 602 remains extending through the entirety of lumen 614, extending distally beyond the distal portion 613 of the tube member 541.

A difference between the configuration of guide extension catheter 500 as shown in FIG. 6C and the configuration of the guide extension catheter as shown in FIG. 6D is that as illustrated in FIG. 6D, the outer delivery cover 324 has been removed from its position encircling the elongate sheath 312, which thereby allows the coil loops 320A-B to urge and expand the elongate sheath 312 from the compressed configuration to an expanded configuration, and in the process opening up the lumen 614 extending through the longitudinal axis encircled by elongate sheath 312 and the coil loops 320A-B from the distal to the proximal ends of the tube member 541.

In various embodiments, the outer delivery cover 324 may be removed by applying a pulling force in a proximal direction on elongate shaft 334 (FIG. 6C), thereby causing the outer delivery cover 324 to be extracted from encircling the tube member 541. In various embodiments, the outer delivery cover 324 is completely removed from the lumen 603 of the guide catheter 604 by pulling elongate shaft 334 proximally over guidewire 602 until the outer delivery cover 324 is extracted through the proximal end of the guide catheter 604. Elongate shaft 310 remains coupled to one or more components of the tube member 541 and extends proximally within the lumen 603 of the guide catheter 604, the elongate shaft 310 having a proximal end that extends out of and beyond a proximal end of the guide catheter. The extension of the elongate shaft 310 out and beyond the proximal end of the guide catheter 604 allows an operator, such as a physician, to control and maintain the position of the tube member 541 and the catheter 500 during various procedures, such as the removal of the outer delivery cover 324 from the tube member, during procedures involving the advancement and retraction of one or more treatment devices through the catheter 500, and including the retrieval of the catheter 500 from the guide catheter 604 once the procedure(s) being performed on the patient have been completed.

In various embodiments, as shown in FIG. 6D, the proximal portion 612 of the guide extension catheter 500 that remains positioned with the lumen 603 of the guide catheter 604 may be expanded so that the outside surfaces of the elongate sheath 312 are in contact, around at least some or all of the perimeter of these outer surfaces, the inner surface(s) forming the lumen 603 within the guide catheter 604. In various embodiments, and as also shown in FIG. 6D, the distal portion 613 of the tube member 541 that remain positioned beyond the distal end 605 of the guide catheter 604 may be in an expanded configuration. In various embodiments, when in this expanded configuration the outside surfaces of the elongate sheath 312 in the distal portion 613 may be in contact, around at least some or all of the perimeter of these outer surfaces, the inner surface(s) of a vascular of a patient where the distal portion 613 is located, including being in contact with a vascular that is located distally beyond the ostium of the patient. In various embodiments, a value for an internal dimension, such as a diameter 611 in cross-section of the lumen 610 of the distal portion 613 when the tube member 541 is in the expanded configuration as shown in FIG. 6D is larger than an internal dimension, such as a diameter 616 in cross-section of the lumen 603 of the guide catheter 604.

In various embodiments, when in the expanded configuration one or more of the loops 320A-B that are positioned in the distal portion 613 may be extended to lie in a plane that is perpendicular or nearly perpendicular to the line of axis of the push member 310, which may provide a maximum dimension 611 in cross-section available for the portion of lumen 614 that is located in the distal portion 613. In various embodiments, when in the expanded configuration one or more of the loops 320A-B that are positioned in the proximal portion 612 may remain partially radially compressed in a direction toward the axis of push member 310 so that these loops lie a plane that is tilted an angle less than ninety degrees relate to the line of axis of the push member 310. As a result, a value for an internal dimension, such as a diameter 617 in cross-section of the portion of the lumen 614 positioned within proximal portion 612 when the guide extension catheter 500 is in the expanded configuration as shown in FIG. 6D may be smaller than a dimension 616 for the lumen 603 of the guide catheter 604. In various embodiments, a diameter 616 of the lumen 603 of the guide catheter 604 near distal end 605 may vary, a diameter 617 of the portion of the lumen 614 of the tube member 541 that is located within the proximal portion 612 may also vary, and a diameter 611 of the portion of lumen 614 of the tube member 541 that is located within the distal portion 613 may vary.

FIG. 6E illustrates a partial cutaway view of the guide extension catheter 500 of FIG. 5 deployed in a guide catheter 604 and receiving a treatment device 620, according to various embodiments. Guide extension catheter 500 as illustrated in FIG. 6E is in the expanded configuration and in the same or approximately the same position as was illustrated and described above with respect to FIG. 6D. As shown in FIG. 6E, a treatment device 620 coupled to a treatment device push member 622 has been advanced through the lumen 603 of the guide catheter 604, through the portion of the lumen 614 of the tube member 541 that is located within the proximal portion 612, and into position within the portion of lumen 614 that is located within the distal portion 613 using guidewire 602. Guidewire 602 remains extending distally from lumen 614. The treatment device 620 may be further advanced out of the distal end of the tube member 541 and to a treatment site within a vascular of a patient (not shown in FIG. 6E, but for example stenotic lesion 218, FIG. 2), in order to provide treatment or other procedures at the treatment site. Because the distal portion 613 of the tube member 541 may extend further into a vascular than would be possible or practical for the distal end of the guide catheter, the delivery of the treatment device when exiting the guide extension catheter may be closer to the location of the treatment site, thus reducing the chances of damage to the vascular wall proximal to the treatment site potentially caused by the advancement of the treatment device done in the absence of the guide extension catheter. Further, the presence of the proximal cap 501 position at the proximal entrance of lumen 614 may provide additional stability to the entrance of the lumen, thereby making it for the leading portions of the treatment devices to be received into lumen 614.

Further, the positioning of the distal portion 613 of the tube member 541 of guide extension catheter being extended to a further location distally within the vascular provides additional stability for the distal end 605 of the guide catheter 604, thereby lessening the possibility of dislodging the distal end 605 of the guide catheter from the vascular structure, such as the ostium, during the time the treatment device 620 is being advanced toward the treatment site and/or during the treatment and/or the procedure(s) being performed on or at the treatment site. In addition, because the coil loops 320A-B remain encapsulated within the layers of the elongate sheath 312 when the tube member 541 is in the expanded configuration, the possibility of the treatment device 620 and/or the push member 622 getting caught on any of the coil loops 320A-N is reduced or completely eliminated. Upon completion of any treatment or other procedures utilizing the treatment device 620, the treatment device may be retrieved back into the lumen 614 utilizing a proximally exerted pulling force applied to push member 622, wherein the treatment device 620 may be fully extracted out the proximal end of guide catheter 604. Upon completion of any treatment or other procedures utilizing the treatment device 620 and removal of the treatment device from the guide catheter 604, the tube member 541 may also be retrieved back through lumen 603 of the guide catheter 604 utilizing a proximally exerted pulling force applied to elongate shaft 310, wherein the treatment device tube member 541 may be fully extracted out the proximal end of guide catheter 604 utilizing elongate shaft 310 for the retrieval.

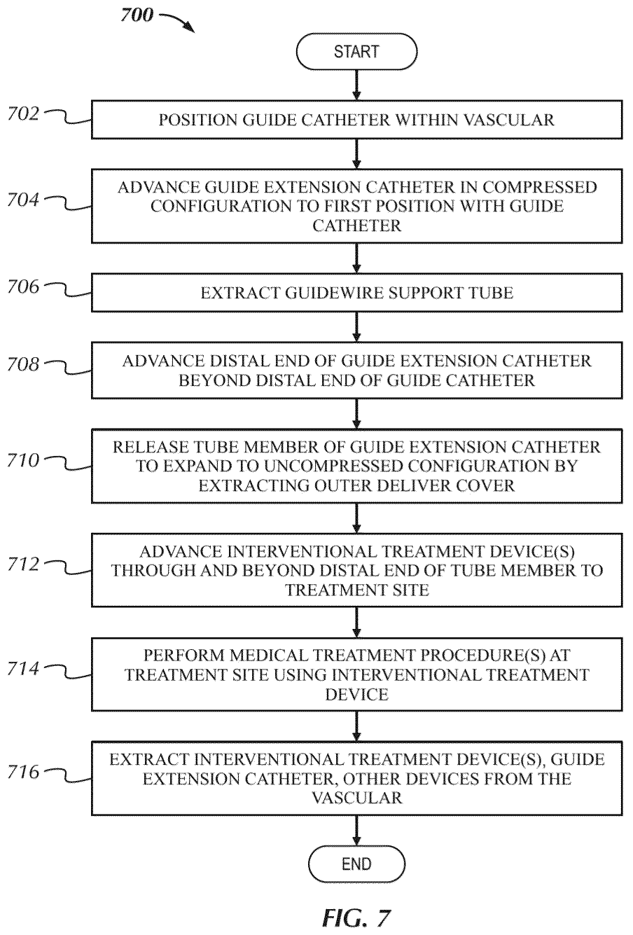

FIG. 7 illustrates a method 700 for accessing a coronary artery, shielding the artery from abrasion or injury, and providing treatment to the artery.

Embodiments of method 700 include positioning an outer catheter, such as a guide catheter, within a vascular of patient (block 702). The outer or guide catheter may include providing a guide catheter formed of polyurethane, for example, and can be shaped along its distal portion to facilitate advancement of a distal end of the outer or guide catheter to a coronary ostium (or other region of interest within a patient's body). Any sized guide catheter may be provided, e.g., 6 F, 7 F, or 8 F guide catheter, where F is an abbreviation for the French catheter scale (a unit to measure catheter diameter (1 F=⅓ mm)). Advancement of the outer or guide catheter so that the distal end of the guide catheter is positioned at a desired region of interest, such as but not limited to the coronary ostium of the patient, may be accomplished by advancing the guide catheter over a guidewire that extends through the vascular and near to, proximate, or past the region of interest.

Embodiments of method 700 include advancing a guide extension catheter in a compressed configuration to a first position within the guide catheter (block 704). In various embodiments, the guide extension catheter is guide extension catheter 300 (FIG. 3), or any equivalent thereof, having an elongated sheath formed and compressed around a guidewire support tube by an outer delivery cover, and having no rigid end cap(s) directly coupled to the elongate sheath. In various embodiments, the guide extension catheter is guide extension catheter 500 (FIG. 5), or any equivalent thereof, having an elongate sheath formed and compressed around a guidewire support tube by an outer delivery cover, and having a rigid end cap(s) directly coupled to a proximal end of the elongate sheath. In various embodiments, advancing a guide extension catheter to the first position within the guide catheter includes receiving at a passageway extending through the guidewire support tube the guidewire that is in place through the guide catheter, and advancing the tube member including the guidewire support tube over the guidewire to the first position. In various embodiments, the first position is defined as having at least the distal end and some portion of the tube member of the guide extension catheter positioned within a lumen of the guide catheter at the proximal end of the guide catheter, and having the guide extension catheter still retained in the compressed configuration.

Embodiments of method 700 include extracting the guidewire support tube (block 706). Extracting the guidewire support tube may include pulling the guidewire support tube proximally away from the tube member of the guide extension catheter so that the guidewire support tube no longer occupies a lumen extending through the elongate sheath of the tube member. In various embodiments, extracting the guide extension tube further includes retrieving the guidewire support tube from the guide catheter by extracting the guidewire support tube through a proximal end of the guide catheter. In various embodiments, extraction of the guidewire support tube is accomplished by applying a force in a proximal direction on the guidewire tube member itself and/or exerting a force in a proximal direction on an elongate shaft coupled to the guidewire support tube and extending proximally from the guidewire support tube to and/or beyond the proximal end of the guide catheter.

Embodiments of method 700 include advancing a distal end of the tube member of the guide extension catheter to and beyond a distal and of the guide catheter (block 708). In advancing the tube member beyond the distal end of the guide catheter, only a portion of the total length dimension of the tube member is advanced beyond the distal end of the guide catheter, while the remaining portion of the tube member remains positioned proximal of the distal end of the guide catheter and remains located within the lumen of the guide catheter. The distal portion of the tube member that extends beyond the lumen of the guide catheter may be referred to as the “distal portion” of the tube member, while the portion of the tube member that remains located proximal of the distal end of the guide catheter and within the lumen of the guide catheter may be referred to as the “proximal portion” of the tube member.

When positioned to partially extend beyond the distal end of the guide catheter, both the distal portion and the proximate portion of the tube member remain, at least initially, in the compressed configuration, wherein both portions of the tube member remain encircled by the outer delivery cover. In various embodiments, when the guide extension catheter is advanced through the guide catheter to a position where at least a portion of the tube member extends distally beyond a distal end of the guide catheter, at least some length of the distal portion of the tube member will extend into the coronary artery of a patient, in some embodiments extending past the ostium. In some embodiments, the guide extension catheter is advanced by providing an advancement force on the push member or elongate shaft coupled to the tube member.

Embodiments of method 700 include releasing the tube member of the guide extension catheter to allow the tube member to expand from the compressed configuration by extracting the outer delivery cover (block 710). In various embodiments, the outer delivery cover is extracted by applying a proximal force to an elongate shaft coupled to the outer delivery cover, the elongate shaft extending proximally beyond the proximal end of the guide catheter, the proximal force adequate to remove the outer delivery cover in a proximal direction away from the tube member. In various embodiments, the outer delivery cover comprises a perforation or other weakness built into the outer delivery cover configured to tear or rip when the proximal force from the elongate shaft that is coupled to the outer delivery cover is transferred to the outer delivery cover, thereby allowing the outer delivery cover to be more easily separated from the tube member. In various embodiments, the elongate shaft remains coupled to the outer delivery cover after the outer delivery cover has been extracted from the tube member, and wherein the elongate shaft is further retracted in a proximal direction through the lumen of the guide catheter to completely remove the elongate shaft and the outer delivery cover from the guide catheter.

Upon removal of the outer delivery cover from the tube member, the radially compressive forces previously exerted on the coil loops and the elongate sheath of the tube member are no longer present, and as a result the coil loops exert spring forced on the elongate sheath, which in turn cause the elongate sheath to expand outward radially from the longitudinal axis of the tube member. This expansion results in the opening of the lumen extending through the elongate sheath, thereby forming a passageway configured to receive and to allow the passage of one or more medical devices through the lumen of the tube member. The tube member may now be referred to as being in the uncompressed configuration.

For the proximal portion of the tube member that is in the uncompressed configuration but still remains located within the lumen of the guide catheter, the outer surfaces of the elongate sheath may expand outward radially to come in contact with the inner surface of the lumen of the guide catheter. For the distal portion of the tube member that is in the uncompressed configuration is positioned beyond the distal end of the guide catheter and outside of the lumen of the guide catheter, the outer surfaces of the elongate sheath may expand outward radially to come in contact an inner wall of an artery located adjacent to the distal portion.

Both the contact of the proximal portion of the guide extension catheter with the inner surface of the guide catheter and/or the contact of the distal portion of the guide extension catheter with an inner wall of an artery can provide additional stability with respect to the positioning of the guide extension catheter, especially when one or more interventional treatment devices are being advanced through, operated, and retrieved through the tube member. Upon removal of the outer delivery cover from the tube member, an elongate shaft or “push member” remains coupled to the tube member and extends proximally beyond the proximal end of the guide catheter, which allows for both control of movements and/or stabilize positioning control over the tube member using forced applied to the elongate shaft and thereby coupled to the tube member. In addition, upon removal of the outer delivery cover from the tube member, the guidewire that extends through the lumen of the guide catheter remains in place and remains extended beyond the distal end of the distal portion of the tube member, providing a mechanism to advance interventional treatment devices to be advanced to, extended through, and retrieve back through the passageway of the lumen extending through the tube member.

Embodiments of method 700 include advancing interventional treatment device(s) through and beyond the distal end of the tube member to a treatment site (block 712). In various embodiments, advanced interventional treatment device(s) through and beyond the distal end of the tube member to a treatment site includes feeding the interventional treatment device(s) over the guidewire extending through the lumen of the guide catheter and the tube member of the guide extension catheter by applying a force in a distal direction to an medical device push member coupled to one or more of interventional treatment device(s) until the interventional treatment device(s) is advanced distally out of the tube member and arrives at the treatment site. In some embodiments, advancing the interventional cardiology device(s) into and through the lumen defined by the tubular member includes protecting an endothelium layer of the coronary artery from injury between the distal end of the guide catheter and a target tissue treatment area. In some embodiments, advancing the interventional cardiology device into and through the lumen defined by the tubular membrane includes protecting an endothelium layer of the coronary artery from injury between the distal end of the guide catheter and a target tissue treatment area. The types of interventional treatment device(s) that may be advanced through the tube member of the guide extension catheter are not limited to any particular type of interventional treatment device.

Embodiments of method 700 include performing medical treatment procedure(s) at the treatment site using the interventional treatment device(s) that have been advanced through the guide extension catheter to the treatment site (block 714). The type of treatment procedures that may be performed at the treatment site are not limited to any particular procedures, may including any procedures that are appropriate to perform based on the interventional treatment devices that have been advanced to the treatment site through the guide extension catheter.

Embodiments of method 700 include extracting interventional treatment device(s), the guide extension catheter and other devices, from the vascular of the patient upon completion of the treatments to be performed at the treatment site (block 716). In various embodiments, any interventional treatment device(s), may be retrieved first, followed by retrieval of the guide extension catheter, and finally followed by retrieval of any remaining devices, such as the guidewire. Retrieval of the interventional treatment device(s) may include extracting the interventional treatment device(s) back in a proximal direction through the lumen of the guide extension catheter, which may remain in place as partially extending beyond the distal end of the guide catheter. Once the interventional treatment device(s) have been retrieved, the guide extension catheter may next be retrieved proximally though the guide catheter, for example using a proximally exerted force applied to the elongate shaft coupled to the tube member of the guide extension catheter. When the guide extension catheter has been retrieved from the guide catheter, the remaining devices, such as the guide catheter and the guidewire, may be retrieved from the vasculature of the patient.

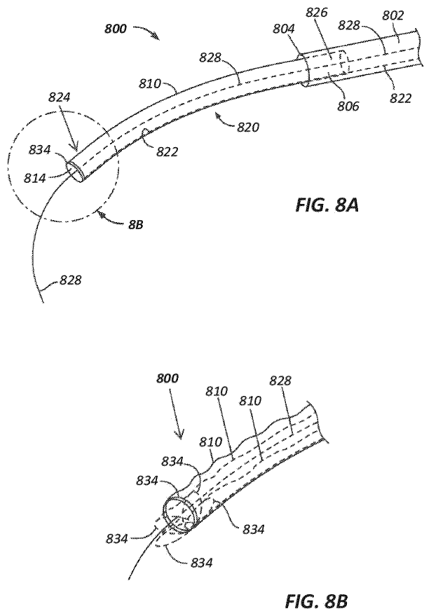

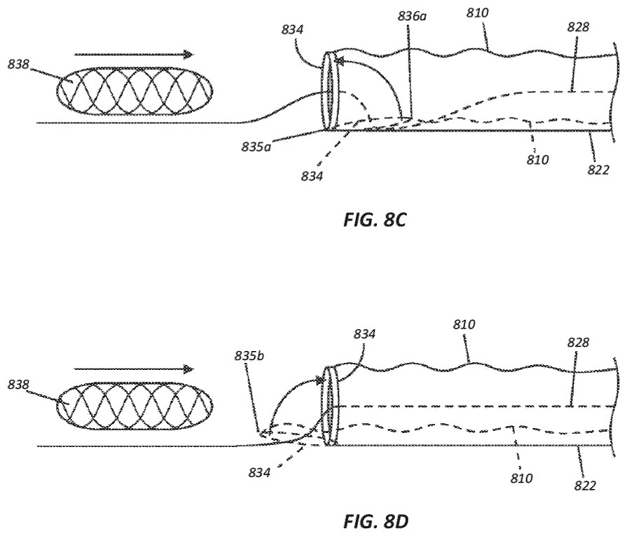

FIG. 8A provides an isometric view of an embodiment of a guide extension catheter 800 featuring a distal reinforcement member configured to pivot, lean, tilt, or otherwise undergo a change in angular position upon interacting with another device advanced or retracted coaxially. Shown extending from a distal end 804 of a guide catheter 802, in this embodiment, the guide extension catheter 800 includes a tube member 820 having a distal end 824, a proximal end 826, and a radially collapsible tubular membrane 810 with a push member 822 affixed thereto. A guidewire 828 is extended through and distally beyond the distal end 824 of the tube member 820, through a lumen 814 of the tubular membrane 810, which is depicted in a fully open/expanded state. The tubular membrane 810 may be freely collapsible and/or foldable in some examples, such that the column strength, radial strength, and/or bend stiffness of the tubular membrane 810 may be non-effective, i.e., wherein the radially collapsible properties of the tubular membrane may have no effective column strength, no effective radial strength, and no effective bend stiffness. In other words, any radial force, column force, or bend force may cause the tubular membrane 810 to deflect, collapse, and/or bend, and wherein the tubular membrane 810 may provide no effective resistance to a radial, column, or bend force. The tubular membrane 810 may have a tensile strength sufficient to prevent tearing of its walls during advancement and withdrawal of an interventional device. For instance, during advancement of an interventional device through the lumen of the tubular membrane 810, the interventional device may provide a force which urges the walls of the tubular membrane 810 radially. The material forming the tubular membrane 810 may have a tensile strength sufficient to withstand the radially outward force of the interventional device such that the interventional device does not tear or otherwise damage the walls of the tubular membrane 810. The tubular membrane 810 may be durably lubricious on one or both of its inner surface or its outer surface via a hydrophobic, silicone, or polymer coating. The lubricious inner surface of the tubular membrane 810 may be configured to reduce friction between the tubular membrane 810 and the interventional device during insertion and/or withdrawal of the device. The lubricious outer surface of the tubular membrane 810 may be configured to reduce friction between the tubular membrane 810 and the guide catheter during operations, such as advancement and/or withdrawal of the guide extension catheter 800 into and/or from an outer or guide catheter. In some embodiments, the cross-sectional diameter of the tube member 820 may vary along the length of the tube member 820 such that, for example, the distal end of the tube member may have a maximum cross-sectional dimension that is greater than that of the proximal end of the push member.

In embodiments, the tube member 820 may not be self-expanding, in whole or in part, such that at least a portion of the radially collapsible tubular membrane 810 may remain in a folded or drooped configuration even when not confined within a guide catheter. According to such embodiments, the passage of interventional devices through the tube member 820 may be necessary for its expansion, such that it otherwise droops or folds around an interventional device being advanced distally therethrough. The guide extension catheter 800 may therefore lack one or more of the features described above in connection with additional embodiments of a guide extension catheter, including one or more support features configured to transition a guide extension catheter from a compressed configuration to an uncompressed configuration. For instance, the tube member 820 may exclude one or more of the above-described coil loops 320 arranged along the length of the tube member 341 included in guide extension catheter 300, and the guide extension catheter 800 may lack one or more of the referenced elongate shafts. One or more components utilized together with guide extension catheter 300, for example, may also be excluded from guide extension catheter 800, non-limiting examples of which may include a guidewire support tube and/or outer delivery cover. One or more of these features may also be included. For example, the push member 822 of guide extension catheter 800 may be similar in one or more respects to the elongate shaft or push member 310 described above. Accordingly, the components the guide extension catheter 800 and their relative arrangement may vary, as may their various properties, features, and functions. In some embodiments, one or more components of the guide extension catheter 800, such as the tube member 820, radially collapsible membrane 810, and/or reinforcement member(s), may resemble one or more components of embodiments of the guide extension catheters described in U.S. Pat. Appln. Pub. No. 2024/0238555 A1, the entire contents of which are incorporated by reference herein.

In the embodiment depicted in FIG. 8A, the proximal end 826 of the tube member 820 may include a first reinforcement member 806, which may comprise a substantially circular component, e.g., a ring, coil, or coil loop. Embodiments of the first reinforcement member 806 may comprise a segment or portion of the device formed from a rigid or semi-rigid, non- collapsible material, which may form a portion of the lumen extending through the tube member 820. Embodiments of the reinforcement member 806 may include or define one or more of an elongate tube, a concave track, a non-elongate tube (e.g., a partial or complete ring or coil), or a combination thereof. Embodiments of the first reinforcement member 806 may comprise various materials, non-limiting examples of which may include one or more metals, plastic, or combinations thereof.

As further shown in FIG. 8A, the distal end 824 of the tube member 820 may include a second, distal reinforcement member 834 comprising a resilient support ring or coil member (hereinafter referred to as “coil member 834”), which may at least partially resemble one or more features of coil loop 320A, for example, such as its form, characteristics, material composition (e.g., shape-memory material, flexible polymer, deformable stainless steel, Nitinol, etc.), and/or relative position along the guide extension catheter 800. The coil member 834 may be attached to the distal end of the tubular membrane 810 and may be configured to facilitate entry, exit, and passage of one or more interventional devices (e.g., treatment catheters) into, beyond, and through the tube member 820. For instance, the coil member 834 may be configured to facilitate entry (or re-entry) of at least a portion of such interventional devices into the tube member 820 during proximal retraction of such devices toward and through the distal end 824 of the tube member 820 from a target site within the vasculature of a subject. The coil member 834 may be configured to facilitate exit of at least a portion of such interventional devices during advancement of such devices through and beyond the distal end 824 of the tube member 820. The coil member 834 may be configured to otherwise facilitate movement of interventional devices during coaxial passage of such devices within, and relative to, the tube member 820. The coil member 834 may comprise a flexible, deformable, shape-memory material and/or resilient configuration that is biased toward a resting state or configuration, which may be an unconstrained configuration.

The resting configuration of the coil member 834 may vary. In some examples, the coil member 834 may be biased toward, or assume, a flat or substantially flat orientation relative to the longitudinal axis of the push member 822 in the resting configuration, such that the coil member 834 defines a small, acute angle with respect to the longitudinal axis of the push member 822 when resting. According to such embodiments, the push member 822 and coil member 834 may, in the resting configuration, define an angle ranging from about 0° to about 80°, inclusive, or any angle therebetween, including less than about 1°, 2°, 3°, 4°, 5°, 10°, 15°, 20°, 25°, 30°, 35°, 40°, 45°, 50°, 55°, 60°, 65°, 70°, 75°, or any angle therebetween. In other embodiments, the coil member 834 may be substantially perpendicular or otherwise orthogonal to a longitudinal axis of a push member 822 in its resting configuration.

When oriented at an orthogonal, e.g., substantially perpendicular, orientation with respect to the longitudinal axis of the push member 822 (which may be defined as being at an angle greater than the small, acute angle referenced in the preceding paragraph), the coil member 834 may establish, expand, and/or maintain a patency of at least a distal portion of the lumen 814 of the tubular membrane 810. In this configuration, for example when not positioned within a guide catheter or confined within an operational space within the vasculature of a subject, the coil member 834 may thus define a distal opening into the tube member 820 oriented and sized to accommodate unobstructed entry and passage of one or more interventional devices therethrough.

In response to contact or interaction with an interventional device, the coil member 834 may tilt, bend, or otherwise lean in a proximal or distal direction, as represented by the dashed outlines of the coil member 834 shown in FIG. 8B. When no interventional device is positioned within the tubular membrane 810, the membrane 810 may be in its collapsed state, loosely draped or folded over the push member 822, which may, in some examples, not be especially conducive to passage of the interventional devices therethrough, in either the proximal or distal direction, for example during retrograde entry of an interventional device from beyond its distal end due to a potential risk of the membrane interfering with the path of the interventional device.

By way of illustration, to facilitate entry of an interventional device being retracted proximally toward the distal end 824 of the tube member 820, contact of the interventional device 838 with the leading edge or distal-most portion of the leaning coil member 834 may cause the coil member 834 to swing, pivot, or tilt to an orientation substantially perpendicular or otherwise angular or orthogonal to the longitudinal axis of the push member 822, thereby transitioning the distal end of the lumen 814 of the tubular membrane 810 to an expanded, non-collapsed state and opening a clear passageway through the opening defined by the coil member 834 and into the tube member 820 through which the interventional device may be retracted proximally (or advanced distally). Accordingly, the angulation of the coil member 834 with respect to the longitudinal axis of the push member 822 may change in response to forces applied by an interventional device, for example during proximal retraction of an interventional device into and through the tube member, or during distal advancement of an interventional device through the coil member. Due to its material composition and configuration, the angulation and/or cross-sectional form of the coil member 834 may also change in response to variations in the vasculature of a subject, e.g., widening and narrowing of tortuous blood vessels.

In embodiments featuring a coil member 834 biased toward a resting configuration in which it leans proximally (see dashed coil member 834 in FIG. 8C), contact between the distal-most portion 835a of the coil member 834 and a proximally retracting interventional device 838 may cause the opposite, “free” end 836a of the coil member 834 to pivot or swing in the direction of the curved arrow shown in FIG. 8C, bringing the attached distal end of the tubular membrane 810 therewith. In embodiments featuring a coil member 834 biased toward a resting configuration in which it leans distally (see dashed coil member 834 in FIG. 8D), contact between the distal-most portion 835b of the coil member 834 (constituting the “free” end in this configuration), and a proximally retracting interventional device 838 may cause the distal-most portion 835b of the coil member 834 to pivot or swing in the direction of the curved arrow shown in FIG. 8D, again bringing the attached distal end of the tubular membrane 810 therewith. Accordingly, proximal retraction of an interventional device may cause the coil member 834 to tilt, lean, or pivot away from the longitudinal axis of the push member 822, such that the cross-sectional area of the lumen 814 of at least a distal portion of the tubular member 810 is increased or maximized, and interference with a proximally retracting interventional device is reduced together with the likelihood of any associated damage to the tubular membrane 810. The angular orientation of the coil member 834 may likewise change in response to movement of a coaxially positioned interventional device in the distal direction relative to the tube member 820, toward a treatment site. The angulation of the coil member 834 may thus be directly responsive to the position and movement of the interventional device 838, along with the dimensions and curvature of the surrounding vessel wall.

Embodiments of the coil member 834 may be integrally formed with the push member 822, such that the coil member 834 may be continuous with the push member 822 and, in some examples, may define the distal end of the push member 822. According to such embodiments, the push member 822 may comprise an elongate push rod or body having a longitudinal axis that aligns substantially with the longitudinal axis of the tube member 820 and guide catheter 802, and a distal portion that coils, curls, or otherwise curves away from its longitudinal axis. In other embodiments, the coil member 834 may comprise a distinct component, portion, or segment affixed, coupled, or otherwise attached to the distal end of the push member 822, for instance via welding.

The present devices, systems, and methods provide or use a delivery tool to reduce arterial injury caused by (i) abrasion of the coronary endothelium during interventional device delivery, or (ii) coronary trauma/dissection caused by large delivery forces, active guide catheter back-up, or relatively rigid guide extension catheters. In contrast to existing more rigid guide extension catheters, the current devices are configured to optimize (1) coronary vessel protection and (2) lubricity of the entire intra-coronary delivery pathway.

The above Detailed Description includes references to the accompanying drawings, which form a part of the Detailed Description. The Detailed Description should be read with reference to the drawings. The drawings show, by way of illustration, specific embodiments in which the present devices, systems, and methods can be practiced. These embodiments are also referred to herein as “examples.”

The Detailed Description is intended to be illustrative and not restrictive. For example, the above-described examples (or one or more features or components thereof) can be used in combination with each other. Other embodiments can be used, such as by one of ordinary skill in the art upon reviewing the Detailed Description and accompanying drawings. Also, various features or components have been or can be grouped together to streamline the disclosure. This should not be interpreted as intending that an unclaimed disclosed feature is essential to any claim. Rather, inventive subject matter can lie in less than all features of a disclosed embodiment. Thus, the following claims are hereby incorporated into the Detailed Description, with each example standing on its own as a separate embodiment as follows:

Embodiment 1. A guide extension catheter, comprising: a proximal elongate shaft; and a distal, self-expanding elongate sheath member coupled with the elongate shaft, the elongate sheath member expandable between a radially contracted configuration and a radially expanded configuration and defining a passageway, the elongate sheath member in the radially contracted configuration having an outer profile no more than three times greater than an outer profile of the elongate shaft, and the elongate sheath member in the radially expanded configuration having an outer profile at least four times greater than the outer profile of the elongate shaft.

Embodiment 2. The guide extension catheter of embodiment 1, further comprising a removable guidewire support tube positioned within the passageway of the elongate sheath member when in the radially contracted configuration and having an inner diameter sized to receive a guidewire therethrough.

Embodiment 3. The guide extension catheter of embodiment 1 or embodiment 2, wherein the elongate shaft is directly coupled with the elongate sheath member.

Embodiment 4. The guide extension catheter of embodiment 3, wherein the elongate shaft is welded to an encapsulated member of the elongate sheath member.

Embodiment 5. The guide extension catheter of any one of embodiments 1 to 4, wherein the elongate sheath member includes a coil or braid and a polymer laminated to the coil or braid.

Embodiment 6. The guide extension catheter of embodiment 5, wherein the coil or braid is formed of a shape memory material.

Embodiment 7. The guide extension catheter of embodiment 5, wherein the coil includes a continuous spiral pattern.

Embodiment 8. The guide extension catheter of embodiment 5, wherein the coil includes a sequence of rings, each ring separated by a connection member that is oriented generally perpendicular to a plane of the ring.

Embodiment 9. The guide extension catheter of embodiment 5, wherein the polymer is configured to expand and contract and includes at least one of ethylene tetrafluoroethylene (ETFE), polytetrafluoroethylene (PTFE), and fluorinated ethylene propylene (FEP).

Embodiment 10. The guide extension catheter of any one of embodiments 1-9, wherein, when the elongate sheath member is in the expanded configuration, the passageway is sized to receive a balloon catheter or a stent catheter.

Embodiment 11. The guide extension catheter of any one of embodiments 1-10, further comprising a removable outer delivery cover surrounding the elongate sheath member when in the radially contracted configuration.

Embodiment 12. The guide extension catheter of embodiment 11, wherein the outer delivery cover includes at least one axially extending line of weakness along which the cover is configured to tear upon removal.

Embodiment 13. The guide extension catheter of embodiment 11, wherein a proximal end of the outer delivery cover includes splitting spreadable handles.

Embodiment 14. The guide extension catheter of embodiment 11, wherein the outer delivery cover has a length less than a combined length of the elongate shaft and the elongate sheath member.

Embodiment 15. A method, comprising: advancing a distal end of a predefined length 6 French guide catheter having a continuous lumen through a blood vessel to an ostium of a coronary artery; advancing a distal end portion of a guide extension catheter through and beyond the distal end of the guide catheter, including advancing an elongate shaft eccentrically coupled with a self-expanding elongate sheath member in a radially contracted configuration through and beyond the distal end of the guide catheter; displacing an outer delivery cover surrounding the elongate sheath member, thereby allowing the elongate sheath member to expand from the radially contracted configuration to a radially expanded configuration defining a passageway having a diameter greater than or equal to 0.056 inches; and while maintaining the distal end portion of the guide extension catheter beyond the distal end of the guide catheter, advancing an interventional device at least partially through the continuous lumen of the guide catheter, into and through the passageway of the elongate sheath member, and into the coronary artery.

Embodiment 16. The method of embodiment 15, wherein advancing the elongate sheath member through and beyond the distal end of the guide catheter in the radially contracted configuration includes receiving a guidewire through a guidewire support tube positioned within the passageway of the elongate sheath member when in the radially contracted configuration.

Embodiment 17. The method of embodiment 16, further comprising removing the guidewire support tube from the passageway prior to advancing the distal end portion of the guide extension catheter through and beyond the distal end of the guide catheter.

Embodiment 18. The method of any one of embodiments 15-17, wherein advancing the elongate sheath member through and beyond the distal end of the guide catheter in the radially contracted configuration includes protecting an endothelium layer of the coronary artery between the distal end of the guide catheter and a target tissue treatment area.

Embodiment 19. The method of embodiment 15, wherein displacing the outer delivery cover includes tearing the outer delivery cover along at least one axially extending line of weakness.

Embodiment 20. The method of any one of embodiments 15-19, wherein displacing the outer delivery cover includes proximally withdrawing the outer delivery cover relative to the elongate sheath member.

Embodiment 21. The method of any one of embodiments 15-20, wherein the outer delivery cover has an inner diameter of 0.042 inches or less.

Embodiment 22. The method of any one of embodiments 15-21, wherein, in the radially expanded configuration, an outer surface of the elongate sheath member is radially biased against an inner surface of the guide catheter at least along its proximal end portion.

Embodiment 23. The method of any one of embodiments 15-22, wherein the elongate sheath member includes a coil or braid formed of a shape memory material, and wherein a fully expanded diameter of the coil or braid is greater than a diameter of the inner surface of the guide catheter.

Embodiment 24. A guide extension catheter positionable within a guide catheter and configured to receive an interventional device for insertion into vasculature includes a push member and a first reinforcement member in contact with the push member. The guide extension catheter also includes a radially-collapsible, tubular membrane in contact with the push member and the first reinforcement member, the tubular membrane positioned distal to the first reinforcement member and collapsed or wrapped about the push member prior to receiving the interventional device. The guide extension catheter further includes a second reinforcement member at a distal end of the radially-collapsible, tubular membrane, the second reinforcement member biased toward a resting configuration and configured to undergo a change in angular position upon interacting with the interventional device advancing or retracting coaxially.

Embodiment 25. The guide extension catheter of embodiment 24, wherein in the resting configuration, the second reinforcement member defines an acute angle with respect to a longitudinal axis of the push member.

Embodiment 26. The guide extension catheter of embodiment 24 or 25, where in a non- resting configuration caused by interacting with the interventional device, the second reinforcement member defines an opening into a lumen of the radially-collapsible, tubular member membrane sized and configured to accommodate entry and passage of the interventional device therethrough

Embodiment 27. The guide extension catheter of any one of embodiments 24-26, wherein the tubular membrane has no effective column strength, no effective radial strength, no independent bend stiffness, and a tensile strength sufficient to prevent tearing during insertion of the interventional device.

Certain terms are used throughout this patent document to refer to features or components. The scope of the present devices, systems, and methods should be determined with reference to the appended claims, along with the full scope of equivalents to which such claims are entitled. In the appended claims, the terms “including” and “in which” are used as the plain- English equivalents of the respective terms “comprising” and “wherein.” Also, in the following claims, the terms “including” and “comprising” are open-ended; that is, a device, system, or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The drawings are provided for the purpose of illustrating example embodiments. The scope of the claims and of the disclosure are not necessarily limited to the systems, apparatus, methods, or techniques, or any arrangements thereof, as illustrated in these figures. In the drawings and description that follow, like parts are typically marked throughout the specification and drawings with the same or coordinated reference numerals. The drawing figures are not necessarily to scale. Certain features of the invention may be shown exaggerated in scale or in somewhat schematic form, and some details of conventional elements may not be shown in the interest of clarity and conciseness.

The Abstract is provided to allow the reader to quickly ascertain the nature of the technical disclosure. It is submitted with the understanding that it will not be used to interpret or limit the scope or meaning of the claims.

What is claimed is:

1. A guide extension catheter, comprising:
a proximal elongate shaft; and
a distal, self-expanding elongate sheath member coupled with the elongate shaft, the elongate sheath member expandable between a radially-contracted configuration and a radially-expanded configuration and defining a passageway,
the elongate sheath member in the radially-contracted configuration having an outer profile no more than three times greater than an outer profile of the elongate shaft, and
the elongate sheath member in the radially-expanded configuration having an outer profile at least four times greater than the outer profile of the elongate shaft.

2. The guide extension catheter of claim 1, further comprising a removable guidewire support tube positioned within the passageway of the elongate sheath member when in the radially-contracted configuration and having an inner diameter sized to receive a guidewire therethrough.

3. The guide extension catheter of claim 1, wherein the elongate shaft is directly coupled with the elongate sheath member.

4. The guide extension catheter of claim 3, wherein the elongate shaft is welded to an encapsulated member of the elongate sheath member.

5. The guide extension catheter of claim 1, wherein the elongate sheath member includes a coil or braid and a polymer laminated to the coil or braid.

6. The guide extension catheter of claim 5, wherein the coil or braid is formed of a shape memory material.

7. The guide extension catheter of claim 5, wherein the coil includes a continuous spiral pattern.

8. The guide extension catheter of claim 5, wherein the coil includes a sequence of rings, each ring separated by a connection member that is oriented generally perpendicular to a plane of the ring.

9. The guide extension catheter of claim 5, wherein the polymer is configured to expand and contract and includes at least one of ethylene tetrafluoroethylene (ETFE), polytetrafluoroethylene (PTFE), and fluorinated ethylene propylene (FEP).

10. The guide extension catheter of claim 1, wherein, when the elongate sheath member is in the expanded configuration, the passageway is sized to receive a balloon catheter or a stent catheter.

11. The guide extension catheter of claim 1, further comprising a removable outer delivery cover surrounding the elongate sheath member when in the radially-contracted configuration.

12. The guide extension catheter of claim 11, wherein the outer delivery cover includes at least one axially-extending line of weakness along which the cover is configured to tear upon removal.

13. The guide extension catheter of claim 11, wherein a proximal end of the outer delivery cover includes splitting spreadable handles.

14. The guide extension catheter of claim 11, wherein the outer delivery cover has a length less than a combined length of the elongate shaft and the elongate sheath member.

15. A method, comprising:
advancing a distal end of a predefined length 6 French guide catheter having a continuous lumen through a blood vessel to an ostium of a coronary artery;
advancing a distal end portion of a guide extension catheter through and beyond the distal end of the guide catheter, including advancing an elongate shaft eccentrically coupled with a self-expanding elongate sheath member in a radially-contracted configuration through and beyond the distal end of the guide catheter;
displacing an outer delivery cover surrounding the elongate sheath member, thereby allowing the elongate sheath member to expand from the radially-contracted configuration to a radially-expanded configuration defining a passageway having a diameter greater than or equal to 0.056 inches; and
while maintaining the distal end portion of the guide extension catheter beyond the distal end of the guide catheter, advancing an interventional device at least partially through the continuous lumen of the guide catheter, into and through the passageway of the elongate sheath member, and into the coronary artery.

16. The method of claim 15, wherein advancing the elongate sheath member through and beyond the distal end of the guide catheter in the radially-contracted configuration includes receiving a guidewire through a guidewire support tube positioned within the passageway of the elongate sheath member when in the radially-contracted configuration.

17. The method of claim 16, further comprising removing the guidewire support tube from the passageway prior to advancing the distal end portion of the guide extension catheter through and beyond the distal end of the guide catheter.

18. The method of claim 15, wherein advancing the elongate sheath member through and beyond the distal end of the guide catheter in the radially-contracted configuration includes protecting an endothelium layer of the coronary artery between the distal end of the guide catheter and a target tissue treatment area.

19. The method of claim 15, wherein displacing the outer delivery cover includes tearing the outer delivery cover along at least one axially-extending line of weakness.

20. The method of claim 15, wherein displacing the outer delivery cover includes proximally withdrawing the outer delivery cover relative to the elongate sheath member.

21. The method of claim 15, wherein the outer delivery cover has an inner diameter of 0.042 inches or less.

22. The method of claim 15, wherein, in the radially-expanded configuration, an outer surface of the elongate sheath member is radially biased against an inner surface of the guide catheter at least along its proximal end portion.

23. The method of claim 22, wherein the elongate sheath member includes a coil or braid formed of a shape memory material, and wherein a fully-expanded diameter of the coil or braid is greater than a diameter of the inner surface of the guide catheter.

24. A guide extension catheter positionable within a guide catheter and configured to receive an interventional device for insertion into vasculature, the guide extension catheter comprising:
a push member;
a first reinforcement member in contact with the push member;
a radially-collapsible, tubular membrane in contact with the push member and the first reinforcement member, the tubular membrane positioned distal to the first reinforcement member and collapsed or wrapped about the push member prior to receiving the interventional device; and
a second reinforcement member at a distal end of the radially-collapsible, tubular membrane, the second reinforcement member biased toward a resting configuration and configured to undergo a change in angular position upon interacting with the interventional device advancing or retracting coaxially.

25. The guide extension catheter of claim 24, wherein in the resting configuration, the second reinforcement member defines an acute angle with respect to a longitudinal axis of the push member.

26. The guide extension catheter of claim 24, wherein in a non-resting configuration caused by interacting with the interventional device, the second reinforcement member defines an opening into a lumen of the radially-collapsible, tubular member membrane sized and configured to accommodate entry and passage of the interventional device therethrough.

27. The guide extension catheter of claim 24, wherein the tubular membrane has no effective column strength, no effective radial strength, no independent bend stiffness, and a tensile strength sufficient to prevent tearing during insertion of the interventional device.
